(19) United States
(12) Patent Application Publication
(10) Pub. No.: US 2026/0269062 A1
(43) Pub. Date: Sep. 10, 2026

(54) Cohort Selection for Treatment Evaluation

(71) Applicant: Georgetown University, Washington, DC (US)

(72) Inventor: Ophir Frieder, Chevy Chase, MD (US)

(21) Appl. No.: 19/373,285

(22) Filed: Oct. 29, 2025

Related U.S. Application Data
(63) Continuation-in-part of application No. 18/986,400, filed on Dec. 18, 2024, now Pat. No. 12,482,568.
Application No. 18/986,400 is a continuation of application No. 18/523,482, filed on Nov. 29, 2023, now Pat. No. 12,211,623.
Application No. 18/523,482 is a continuation of application No. 17/852,521, filed on Jun. 29, 2022, now Pat. No. 11,869,664.
Application No. 17/852,521 is a continuation of application No. 17/555,675, filed on Dec. 20, 2021, now Pat. No. 11,410,763.
Application No. 17/555,675 is a continuation of application No. 17/088,172, filed on Nov. 3, 2020, now Pat. No. 11,238,966.
(60) Provisional application No. 62/930,072, filed on Nov. 4, 2019.
(60) Provisional application No. 63/042,676, filed on Jun. 23, 2020.

(51) Int. Cl. G16H 50/20; G06F 9/54; G06F 18/24; G06N 7/01; G16H 20/10; G16H 50/80; G16H 70/40

(52) U.S. Cl. CPC G16H 50/20; G06F 9/545; G06F 18/24; G06N 7/01; G16H 20/10; G16H 50/80; G16H 70/40

(57) ABSTRACT

A computer-implemented method for selecting patient cohorts for clinical trials that retrieves candidate medical records from an electronic medical repository and analyzes historical patient data for an initial population of candidate patients eligible for the clinical trial. The method identifies adverse effect patient characteristics associated with severe adverse effects (SAE) or excessive adverse effects (EAE) for a proposed treatment and removes candidate patients having such characteristics. The method further identifies and removes patients having negative outcome patient characteristics associated with treatment failure, while identifying positive outcome patient characteristics associated with treatment success. The method returns a subset of eligible candidate patients based on positive outcome characteristics while excluding those with adverse effect or negative outcome characteristics.

### CROSS-REFERENCE TO RELATED APPLICATIONS

This patent application is a continuation-in-part of U.S. patent application Ser. No. 18/986,400 filed Dec. 18, 2024, which is a continuation of U.S. patent application Ser. No. 18/523,482 filed Nov. 19, 2023, which is a continuation of U.S. patent application Ser. No. 17/852,521, filed Jun. 29, 2022 (now U.S. Pat. No. 11,869,664 issued Jan. 9, 2024), which is a continuation of U.S. patent application Ser. No. 17/555,675, filed Dec. 20, 2021 (now U.S. Pat. No. 11,410,763, issued Aug. 9, 2022, is a continuation of U.S. patent application Ser. No. 17/088,172, filed Nov. 3, 2020 (now U.S. Pat. No. 11,238,966, issued Feb. 1, 2022), which claims the benefit of U.S. Provisional Application No. 62/930,072, filed Nov. 4, 2019 and claims the benefit of U.S. Provisional Application No. 63/042,676, filed Jun. 23, 2020, the contents of all of which are incorporated herein in their entirety.

### BACKGROUND

The present disclosure relates to systems and methods that may provide techniques to predict the success or failure of a drug used for disease treatment using an accurate and efficient model to predict the success potential of a specific drug prescription for a given ailment.

Erroneous medication prescription is defined as a failure in the medication treatment process that results in an unsuccessful treatment or harmful outcome to patients. Clinicians have the responsibility to accurately diagnose and adequately treat a patient's disease. For treatments that require medications, the ideal prescription is the one that is most effective and presents the least harmful side effects. Yet, this is not always achieved. Further, for newly emergent diseases, it is important that effective treatments be identified quickly and efficiently.

For example, at present, there are few, if any, fully approved coronavirus treatments. Remdesivir, a new intravenous antiviral, received an FDA emergency use authorization. However, researchers are testing existing medications (targeted for treatment of other conditions) for COVID-19 treatment. Such drugs being tested may include, for example, Remdesivir, a new drug, Dexamethasone, a corticosteroid currently used for autoimmune and allergic reactions, Hydroxychloroquine and Chloroquine. currently used for malaria & autoimmune diseases, Azithromycin, an antibiotic currently used for bronchitis & pneumonia, Tocilizumab (Actemra), currently used for Rheumatic arthritis, Kaletra (lopinavir/ritonavir), currently used as an HIV medication, Tamiflu currently used for Influenza, Colchicine currently used for Gout, etc. Many such medications are used by thousands or millions of patients for whom medical records are available. Further, COVID-19 treatments are cohort specific, with varying guidelines for identifying patients depending on their health. For example, patients with history of cancer, diabetes, digestive and liver health, etc. have different care guidelines. Accordingly, the likelihood of success of a particular drug may vary with patient history and demographics.

The rapid growth of patient Electronic Health Records (EHRs) provides opportunities to develop a data-driven analytical application on medical data. Many approaches for many medical applications exist.

Due to the complex nature of EHR data, implementing a predictive model is difficult. For example, electronic phenotyping is the process of extracting relevant features from EHRs, a major step before performing an analytical task. Such approaches transform EHRs into vector representations via various feature extraction techniques (e.g., electronic phenotyping). The extracted feature vectors, where each dimension corresponds to a certain medical concept, are fed into a linear classifier. This flattening formulation of EHRs ignores temporal relationships between medical events in a patient's history, reducing effectiveness. On the other hand, many extraction tasks require domain medical knowledge to generate hand-crafted features, which is not efficient and cost prohibitive at large scale. Thus, as a feature extraction technique, electronic phenotyping may cause information loss on the discriminant features.

Recently, the emergence of deep learning models pose other ways to analyze EHR data (e.g., EHR data embedding) which achieve better performance with significantly less feature engineering. However, result interpretation of such systems is difficult. For example, Recurrent Neural Networks (RNN) model time series medical data. However, interpretability concerns associated with deep learning approaches, particularly in the medical domain, limit their use. Notwithstanding, the trade-off to achieve high accuracy and high interpretability remains.

Many studies introduce attention-based RNN models to improve interpretability. However, the majority of efforts rely on publicly available datasets or on a collaborating hospital's EHR system where patient demographic information is mostly uniform. Unfortunately, this uniformity of data fails to exist when developing approaches for real-world, integrated EHR systems (e.g., insurance claim-based EHR systems). On this occasion, highly temporally dependent data attributes with high noise and variance often induce model over-fitting. Such a problem may be addressed with a proposed graph-kernel EHR predictive model, yet they only consider a single medication with immediate outcome observations. For chronic diseases, long-term disease progression coupled with EHR complexity complicates the effort. Thus, attention-based deep learning models and handcrafted kernel computations are limited to handle complex EHR under long-term disease progression. The increased divergence and noise on data attributes over-fit the deep learning model and defeat the handcrafted kernel.

Accordingly, a need arises for techniques to predict the success or failure of a drug used for disease treatment that may provide improved accuracy and efficiency and may provide identification of drugs most likely to be effective that are patient-specific as well as disease specific.

In addition, a common challenge in clinical trials is the identification and selection of appropriate patient cohorts. Finding suitable cohorts is both difficult and expensive. Current approaches to cohort selection often rely on basic demographic and medical criteria, without adequately considering the complex relationships between patient characteristics and treatment outcomes. Accordingly, a need arises for techniques to predict and mitigate adverse effects while identifying patients most likely to benefit from a proposed treatment.

### SUMMARY

Embodiments of the present systems and methods may provide techniques to predict the success or failure of a drug used for disease treatment using an accurate and efficient model to predict the success potential of a specific drug prescription for a given ailment. For example, embodiments may predict success or failure of drug prescription by formulating a binary graph classification problem without the need of electronic phenotyping. First, training data may be identified, such as success and failure patients for target disease treatment within a user-defined time period. The set of medical events from patient Electronic Health Records (EHRs) that occur within this time quantum may be extracted. Then, a classification task may be performed on the graphical representation of the patient EHRs. The graphical representation provides an opportunity to model the EHRs in a compact structure with high interpretability.

As disclosed herein, patients need not be human. That is, the methods and systems disclosed herein are applicable to any animal, human or non-human, under clinical care. Embodiments disclosed are exemplified without loss of generality using human patients.

Embodiments of the present systems and methods may provide a kernel based deep architecture to predict the success or failure for drug prescription given to a patient. The success and failure of medications on patients may be identified to provide targeted disease treatment. An EHR prior to the disease diagnosis is included for each patient, and their graphical representation (e.g., patient graph), where nodes denote all medical events with day differences as edge weights, are built. The binary graph classification task is performed directly on the patient graph via a deep architecture. Interpretability is readily available and easily accepted by users without further post-processing due to the nature of the graph structure.

Embodiments of the present systems and methods may provide a novel graph kernel, Temporal proximity kernel, which efficiently calculates temporal similarity between two patient graphs. The kernel function is proven to be positive definite, increasing the model availability by using a kernelized classifier such as Support Vector Machine (SVM). To obtain the multi-view aspect, we combine the temporal proximity kernel with the node kernel and the shortest path kernel as a single kernel through multiple kernel learning.

To perform large scale and noise-resistant learning objectives, embodiments may transfer the original task to similarity-based classification, where each row in the kernel gram matrix is considered as a feature vector with each dimension expressing the similarity measurement with specific training examples. A multiple graph kernel fusion approach is proposed to learn kernel representation in an end-to-end manner for the best kernel combination. We argue that representation learning is a typical kernel approximation which preserves the similarity while reducing the dimension for the original kernel matrix. The embedding weight for each kernel supports the interpretation to the prediction via most similar cases by selecting one or a plurality of top relevant embedding dimension(s).

Embodiments of the present systems and methods may provide a cross-global attention graph kernel network to learn optimal graph kernels on a graphical representation of patient EHRs. The novel cross-global attention node matching automatically captures relevant information in biased long-term disease progression. In contrast to attention-based graph similarity learning that relies on a pairwise comparisons of training pairs or triplets, our matching is performed on a batch of graphs simultaneously by a global cluster membership assignment. This is accomplished without the need to generate training pairs or triplets for pairwise computations and seamlessly combines classification loss. The learning process is guided by cosine distance. The resulting kernel, compared to its Euclidean distance counterpart, has better noise resistance under a high dimension space. Unlike distance metric learning and aforementioned graph similarity learning, we align our learned distance and graph kernel to a classification objective. We formulate an end-to-end training by jointly optimizing contrastive and kernel alignment loss with a Support Vector Machine (SVM) primal objective. Such a training procedure encourages node matching and similarity measurement to produce ideal classification, providing interpretation on prediction. The resulting kernel function can be directly used by an off-the-shelf kernelized classifier (e.g., scikit-learn SVC). The cross-global attention node matching and kernel-based classification makes it interpretable in both knowledge discovery and prediction case study.

Embodiments may provide one-shot disease processing, for example, for an antibiotic medication. To perform one-shot disease processing, a database of medical history data may be partitioned according to disease diagnosis. A suggested medication may be attached to the data and used to predict a likelihood of success or failure of the medication and to identify similar individuals.

Embodiments may provide COVID-19 processing based on a presumptive medication. A database of medical history data may be partitioned according to those having used the presumptive medication. Patient graphs may be retained up until the last presumptive medication use within a surveillance window. A suggested medication may be attached to the data and used to identify similar individuals indicating a likelihood of success or failure of use of the suggested medication for others diseases under consideration.

For example, in an embodiment, a method of determining drug efficacy may be implemented in a computer system comprising a processor, memory accessible by the processor and storing computer program instructions an data, and computer program instructions to perform for a plurality of patients, generating a directed acyclic graph from health related information of each patient, each directed acyclic graph comprising a first node representing first demographic information of the patient, a plurality of additional nodes, each additional node representing a medical event of the patient, at least one first edge connecting the first node to an additional node, the first edge having a weight based on second demographic information of the patient, and a plurality of additional edges, each additional edge connecting nodes representing two consecutive medical events, the edge having a weight based on a time difference between the two consecutive medical events, capturing a plurality of features from each directed acyclic graph, generating a binary graph classification model on captured plurality of features of each directed acyclic graph, determining a probability that a drug or treatment will be effective using the binary graph classification model, and determining a drug to be prescribed to a patient based on the determined probability.

In embodiments, the plurality of features may be captured by transforming each directed acyclic graph to a shortest path graph, generating a temporal topological kernel by recursively calculating similarity among temporal ordering on a plurality of groups of additional nodes, and generating a temporal substructure kernel on additional edges connecting additional nodes in each group of additional nodes. The plurality of features may be captured by generating a topological ordering of each directed acyclic graph based on an order of occurrence of a label associated with each additional node in each directed acyclic graph, generating a topological sequence of each directed acyclic graph comprising a plurality of levels indicating an order of occurrence of a same node label in the topological sequence, generating a temporal signature for each directed acyclic graph comprising a series of total passage times from the first node to each additional node in a union of a plurality of topological sequences, generating a temporal proximity kernel between a plurality of pairs of temporal signatures, generating a shortest path kernel by calculating an edge walk similarity on shortest path graphs for a plurality of pairs of directed acyclic graphs, generating a node kernel by comparing node labels of a plurality of pairs of directed acyclic graphs, and fusing the temporal proximity kernel, the shortest path kernel, and the node kernel. The fusing may comprise reducing dimensions of the temporal proximity kernel, the shortest path kernel, and the node kernel for the plurality of pairs of directed acyclic graphs, and averaging embeddings of the temporal proximity kernel, the shortest path kernel, and the node kernel for the plurality of pairs of directed acyclic graphs. Determining a success or failure of the fusion may comprise using a sigmoid layer.

In an embodiment, a system for determining drug efficacy may comprise a processor, memory accessible by the processor, and computer program instructions stored in the memory and executable by the processor to perform for a plurality of patients, generating a directed acyclic graph from health related information of each patient, each directed acyclic graph comprising a first node representing first demographic information of the patient, a plurality of additional nodes, each additional node representing a medical event of the patient, at least one first edge connecting the first node to an additional node, the first edge having a weight based on second demographic information of the patient, and a plurality of additional edges, each additional edge connecting nodes representing two consecutive medical events, the edge having a weight based on a time difference between the two consecutive medical events, capturing a plurality of features from each directed acyclic graph, generating a binary graph classification model on captured plurality of features of each directed acyclic graph, determining a probability that a drug or treatment will be effective using the binary graph classification model, and determining a drug to be prescribed to a patient based on the determined probability.

In an embodiment, a computer program product for determining drug efficacy may comprise a non-transitory computer readable storage having program instructions embodied therewith, the program instructions executable by a computer, to cause the computer to perform a method that may comprise for a plurality of patients, generating a directed acyclic graph from health related information of each patient, each directed acyclic graph comprising a first node representing first demographic information of the patient, a plurality of additional nodes, each additional node representing a medical event of the patient, at least one first edge connecting the first node to an additional node, the first edge having a weight based on second demographic information of the patient, and a plurality of additional edges, each additional edge connecting nodes representing two consecutive medical events, the edge having a weight based on a time difference between the two consecutive medical events, capturing a plurality of features from each directed acyclic graph, generating a binary graph classification model on captured plurality of features of each directed acyclic graph, determining a probability that a drug or treatment will be effective using the binary graph classification model, and determining a drug to be prescribed to a patient based on the determined probability.

The present disclosure provides computer-implemented methods and systems for selecting patient cohorts for clinical trials. The method involves a systematic approach to “qualify” or optimize clinical trials by analyzing historical patient data to identify patterns associated with adverse effects and treatment outcomes.

In one embodiment, a computer-implemented method for selecting a patient cohort for a clinical trial comprises: retrieving candidate medical records from an electronic medical repository; analyzing historical patient data for an initial population of candidate patients; identifying adverse effect patient characteristics associated with severe adverse effects (SAE) or excessive adverse effects (EAE); removing candidate patients having such adverse effect characteristics; identifying negative outcome patient characteristics associated with treatment failure; removing candidate patients having such negative characteristics; identifying positive outcome patient characteristics associated with treatment success; and returning a subset of eligible candidate patients based on the positive outcome characteristics while excluding those with adverse effect or negative outcome characteristics.

In some embodiments, the disclosure utilizes digital twin analysis, wherein patients with similar medical characteristics are identified and their treatment histories are analyzed to predict outcomes for candidate patients without direct exposure to the proposed treatment. Machine learning models, such as the models described herein, including large language models (LLMs), are trained on patient graph structures to increase prediction accuracy.

### BRIEF DESCRIPTION OF THE DRAWINGS

The details of the present invention, both as to its structure and operation, can best be understood by referring to the accompanying drawings, in which like reference numbers and designations refer to like elements.

FIG. 1 is an exemplary illustration of set of patient EHRs, according to embodiments of the present systems and methods.

FIG. 2 is an exemplary illustration of a Directed Acyclic Graph (DAG), according to embodiments of the present systems and methods.

FIG. 3 is an exemplary illustration of examples of success and failure cases, according to embodiments of the present systems and methods.

FIG. 4 is an exemplary illustration of a predictive framework directed to predicting effectiveness of a prescribed drug, according to embodiments of the present systems and methods.

FIG. 5 is an exemplary flow diagram of a process of transferring the input from patient graphs to temporal signatures, according to embodiments of the present systems and methods.

FIG. 6 is an exemplary flow diagram of a process of transferring the input from patient graphs to a shortest path kernel, according to embodiments of the present systems and methods.

FIG. 7 is an exemplary flow diagram of a process of transferring the input from patient graphs to a node kernel, according to embodiments of the present systems and methods.

FIG. 8 is an exemplary flow diagram of a process of operation of a multiple graph kernel fusion architecture (MGKF) to perform graph classification, according to embodiments of the present systems and methods.

FIG. 9 is an exemplary flow diagram of a process of interpretation, according to embodiments of the present systems and methods.

FIG. 10 is an exemplary illustration of a predictive framework directed to predicting effectiveness of a course of treatment for a chronic condition, according to embodiments of the present systems and methods.

FIG. 11 is an exemplary illustration of a Cross-Global Attention Graph Kernel Network learning an end-to-end deep graph kernel on a batch of graphs, according to embodiments of the present systems and methods.

FIG. 12 is an exemplary illustration of matching by retrieving cluster identity from global node clusters, according to embodiments of the present systems and methods.

FIG. 13 is an exemplary illustration of a patient DAG illustrating success with pneumonia treatment, according to embodiments of the present systems and methods.

FIG. 14 is an exemplary illustration of a patient DAG illustrating failure with pneumonia treatment, according to embodiments of the present systems and methods.

FIG. 15 is an exemplary flow diagram of a process for prediction for outcome of a drug prescription, according to embodiments of the present systems and methods.

FIG. 16 is an exemplary flow diagram of a process of training a classifier with an MGKF framework, according to embodiments of the present systems and methods.

FIG. 17 is an exemplary flow diagram of a portion of a process of using the trained classifiers with an MGKF framework to perform prediction for each type of disease, according to embodiments of the present systems and methods.

FIG. 18 is an exemplary flow diagram of a portion of a process of using the trained classifiers with an MGKF framework to perform prediction for each type of disease, according to embodiments of the present systems and methods.

FIG. 19 is an exemplary flow diagram of a process of predicting drug and/or treatment outcomes, according to embodiments of the present systems and methods.

FIG. 20 is a flowchart diagram showing the systematic process for patient cohort selection, according to embodiments of the disclosure.

FIG. 21 is a schematic diagram depicting a patient population, according to embodiments of the disclosure.

FIG. 22 is a schematic diagram showing selected cohort candidates within the patient population, according to embodiments of the disclosure.

FIG. 23 is a schematic diagram identifying one or more digital twins within the patient population.

FIG. 24 is a schematic diagram illustrating a patient distribution after cohort selection processing, according to embodiments of the disclosure.

FIG. 25 is an exemplary block diagram of a computer system, in which processes involved in the embodiments described herein may be implemented.

### DETAILED DESCRIPTION

Embodiments of the present systems and methods may provide techniques to predict the success or failure of a drug used for disease treatment using an accurate and efficient model to predict the success potential of a specific drug prescription for a given ailment. For example, embodiments may predict success or failure of drug prescription by formulating a binary graph classification problem without the need of electronic phenotyping. First, training data may be identified, such as success and failure patients for target disease treatment within a user-defined time period. The set of medical events from patient Electronic Health Records (EHRs) that occur within this time quantum may be extracted. Then, a classification task may be performed on the graphical representation of the patient EHRs. The graphical representation provides an opportunity to model the EHRs in a compact structure with high interpretability.

Embodiments of the present systems and methods may provide a kernel based deep architecture to predict the success or failure for drug prescription given to a patient. The success and failure of medication on patients are identified for targeted disease treatment to generate are used to define the success and failure cases. An EHR prior to the disease diagnosis is included for each patient, and their graphical representation (e.g., patient graph), where nodes denote all medical events with day differences as edge weights, are built. The binary graph classification task is performed directly on the patient graph via a deep architecture. Interpretability is readily available and easily accepted by users without further post-processing due to the nature of the graph structure.

Embodiments of the present systems and methods may provide a novel graph kernel, Temporal proximity kernel, which efficiently calculates temporal similarity between two patient graphs. The kernel function is proven to be positive definite, increasing the model availability by using a kernelized classifier such as Support Vector Machine (SVM). To obtain the multi-view aspect, we combine the temporal proximity kernel with the node kernel and the shortest path kernel as a single kernel through multiple kernel learning.

To perform large scale and noise-resistant learning objectives, embodiments may transfer the original task to similarity-based classification, where each row in the kernel gram matrix is considered as a feature vector with each dimension expressing the similarity measurement with specific training examples. A multiple graph kernel fusion approach is proposed to learn kernel representation in an end-to-end manner for the best kernel combination. We argue that representation learning is a typical kernel approximation which preserves the similarity while reducing the dimension for the original kernel matrix. The embedding weight for each kernel supports the interpretation to the prediction via most similar cases by selecting top relevant embedding dimension.

Embodiments of the present systems and methods may provide a cross-global attention graph kernel network to learn optimal graph kernels on a graphical representation of patient EHRs. The novel cross-global attention node matching automatically captures relevant information in biased long-term disease progression. In contrast to attention-based graph similarity learning that relies on a pairwise comparisons of training pairs or triplets, our matching is performed on a batch of graphs simultaneously by a global cluster membership assignment. This is accomplished without the need to generate training pairs or triplets for pairwise computations and seamlessly combines classification loss. The learning process is guided by cosine distance. The resulting kernel, compared to its Euclidean distance counterpart, has better noise resistance under a high dimension space. Unlike distance metric learning and aforementioned graph similarity learning, we align our learned distance and graph kernel to a classification objective. An end-to-end training may be formulated by jointly optimizing contrastive and kernel alignment loss with a Support Vector Machine (SVM) primal objective. Such a training procedure encourages node matching and similarity measurement to produce ideal classification, providing interpretation on prediction. The resulting kernel function can be directly used by an off-the-shelf kernelized classifier (e.g., scikit-learn SVC). The cross-global attention node matching and kernel-based classification makes it interpretable in both knowledge discovery and prediction case study.

In embodiments, up to three kernels may be used to achieve multi-view similarity measurement-reducing potential medical record “noise”. For example, some training examples may dominate prediction results due to higher kernel value. Embodiments may incorporate additional kernels to balance this effect to improve prediction. Example of kernels that may be used include a Temporal Proximity Kernel, which may provide temporal ordering and time difference of medical events, a Shortest Path Kernel, which may provide general connectivity of medical events, and a Node Kernel, which may provide general overlapping of medical events.

Embodiments may provide one-shot disease processing, for example, for an antibiotic medication. To perform one-shot disease processing, a database of medical history data may be partitioned according to disease diagnosis. A suggested medication may be attached to the data and used to predict a likelihood of success or failure of the medication and to identify similar individuals.

Embodiments may provide COVID-19 processing based on a presumptive medication. A database of medical history data may be partitioned according to those having used the presumptive medication. Patient graphs may be retained up until the last presumptive medication use within a surveillance window. A suggested medication may be attached to the data and used to identify similar individuals indicating a likelihood of success or failure of use of the suggested medication for others diseases under consideration.

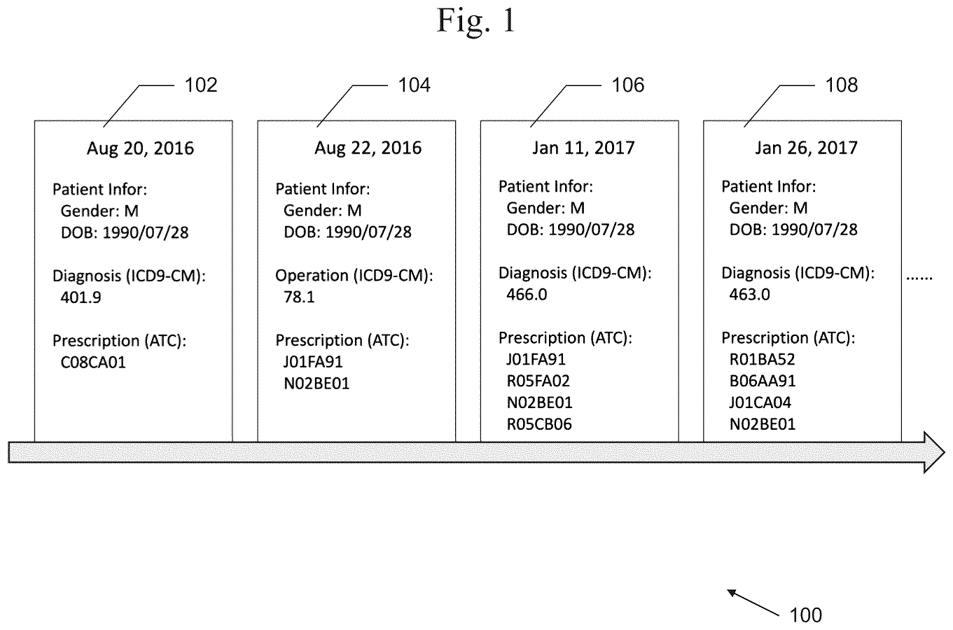

An exemplary set of patient EHRs 100 is shown in FIG. 1. EHRs 100 may include a plurality of records 102-108. Typically, each record relates to a different patient interaction with medical staff, diagnosis, test result, etc., and may include information such as the date of the interaction, diagnosis, test result, etc., demographic information about the patient, such as identity, gender, date of birth, etc., information about the diagnosis or diagnoses, information about the prescription(s), etc.

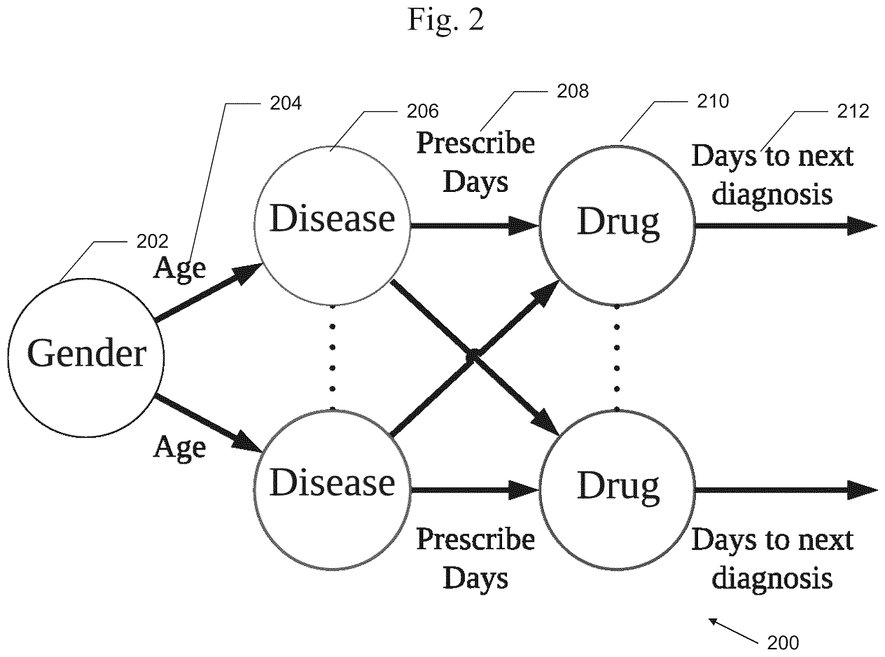

A patient's EHR may be formulated as a Directed Acyclic Graph (DAG) 200, an example of which is shown in FIG. 2. For example, each node represents a medical event, such as a disease diagnosis 206, a drug prescription 210, etc., and an edge between two nodes represents an ordering with the time difference as edge weight (e.g., days). For example, the edge weights may include the prescription day 208, the days to next diagnosis 212, etc. The demographic information of the patient, such as, gender, may be represented as a node 202 that connects to the first medical event 206 with age 204 as an edge weight. In this example, only gender and age are used as demographic information to simplify the model.

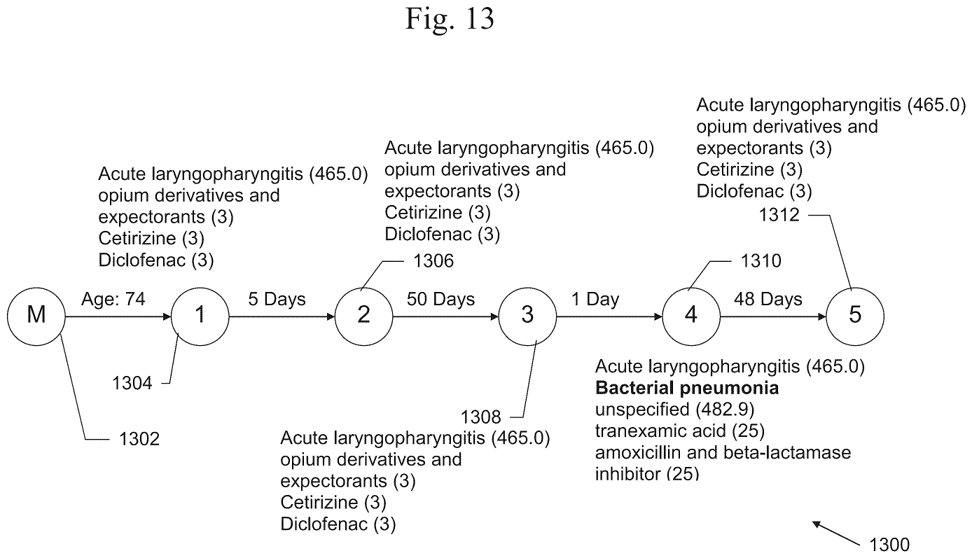

A more detailed example 1300 of a patient DAG illustrating success with pneumonia treatment is shown in FIG. 13. In this example, the diagnosis at each stage 1304-1312 of treatment, as well as the prescriptions at each stage of treatment are shown. This example illustrates a success because there is no diagnosis of pneumonia for more than four weeks after the end of the course of treatment, stage 5 1312.

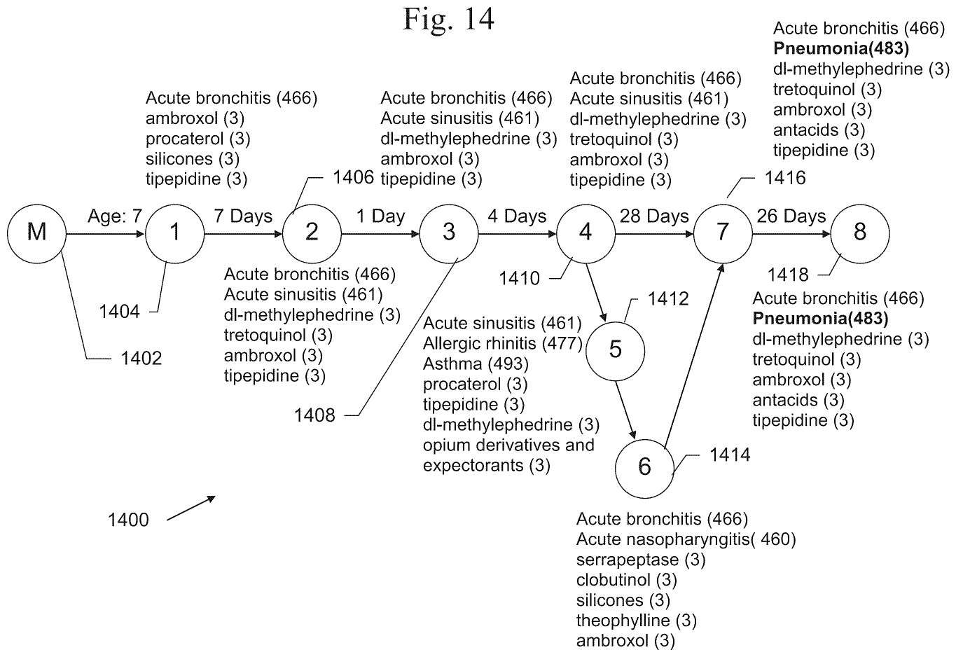

A more detailed example 1400 of a patient DAG illustrating failure with pneumonia treatment is shown in FIG. 14. In this example, the diagnosis at each stage 1404-1418 of treatment, as well as the prescriptions at each stage of treatment are shown. This example illustrates a failure in treatment of pneumonia 1416 because there is a diagnosis of pneumonia within four weeks after a stage of treatment at stage 8 1418.

In embodiments, an EHR graph representation may be defined as follows: Given n medical events, set M={(m₁, t₁), . . . , (mₙ, t₁)} represents a patient's EHR with mᵢ denoting a medical event such as diagnosis, and tᵢ denoting the time for mᵢ. Then the patient graph may be defined as follows: Definition 1 (Patient Graph). The patient graph P_{g}=(V, E) of events M is a weighted directed acyclic graph with its vertices V containing all events mᵢ∈M and edges E containing all pairs of consecutive events (mᵢ, mⱼ). The edge weight from node i to node j is defined as Wᵢⱼ=tⱼ−tᵢ, which defines the time interval between mᵢ, mⱼ.

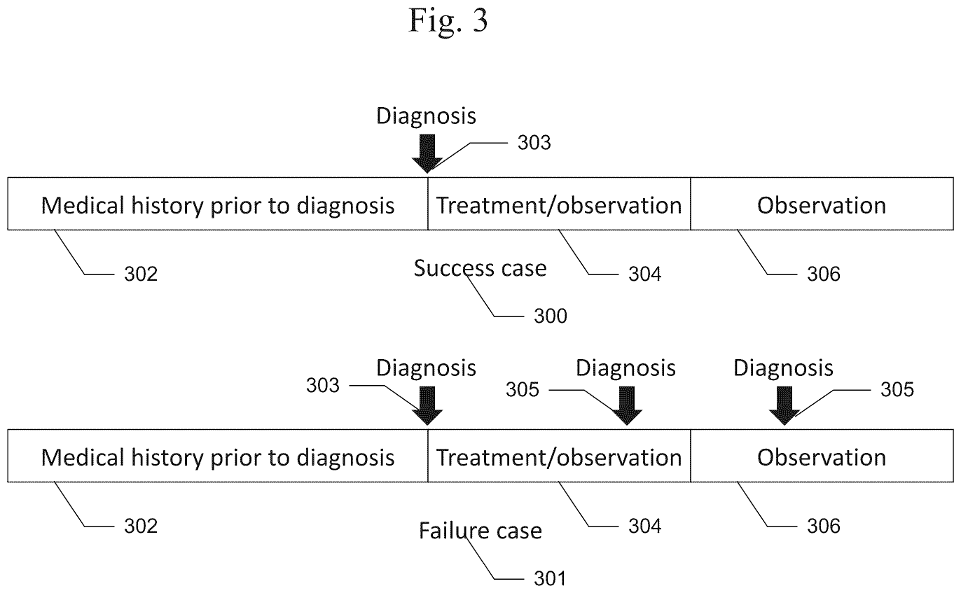

Given a disease diagnosis of a patient, a drug prescription for the diagnosis is considered a failure if the patient has a second same diagnosis within an observation window. Otherwise, the prescription is considered a success. Examples of success 300 and failure 301 cases are shown in FIG. 3. The failure case 301 may be labelled as positive, and the success 300 case may be labelled as negative. To capture historical factors, each case may contain previous medical history events 302 prior to the diagnosis date 303 in a user-defined period. Each case may be treated as a subset of patient EHRs as shown in FIG. 1, which contains a multiple-event single-patient EHR. In short, each case contains the medical events before and after the disease diagnosis for a user-defined period. A drug prescription for the diagnosis is considered a failure if the patient has a second same diagnosis 305 within an observation window, which may include a period of treatment and observation 304, and a period of observation 306.

In embodiments, this may be extended to define the success or failure of treatment plan for a chronic disease, following the guidelines published by the National Medical Association for selected chronic diseases. Generally, an observation window 306 is defined after a treatment period 304 (which may include observation as well) to monitor whether the given treatment plan achieves its treatment objective (such as no severe complication occurrence in 5 years). Given a chronic disease diagnosis, a treatment may be considered a failure if the patient is diagnosed 305 with a selected severe complication or comorbidity within the post treatment observation window 306. Otherwise, the treatment may be considered a success. Due to the chronic disease long-term progression where past factors are potentially decisive, all medical histories may be included prior to the first diagnosis date. Each case may be treated as a set of medical records from a patient's EHR as in FIG. 1. The terms patient and case may be used interchangeably herein.

Given a patient EHR, the patient's current diagnosis, and the drug prescription to the current diagnosis, embodiments may predict the success or failure of a prescribed medication. A temporal graph Gᵢ may be created that consists of the current diagnosis, the drug prescription to the current diagnosis, and the medical events in the patient EHR prior to the current diagnosis. Then a binary graph classification problem may be formulated on the resulting temporal graph by considering the following dual optimization problem for a Support Vector Machine (SVM):

$${maximize}_{α} {\sum }_{i} {α}_{i} - \frac{1}{2} {\sum }_{j , k} {α}_{i} {α}_{k} {y}_{i} {y}_{k} K ( {G}_{j} , {G}_{k} ) \qquad ( 1 b )$$

$$subject to 0 \leq {α}_{i} \leq C , i = 1 , … , N \qquad ( 1 a )$$

$${\sum }_{i} {α}_{i} {y}_{i} = 0 , i = 1 , … , N \qquad ( 1 c )$$

where K is a positive definite graph kernel on input graphs Gⱼ, Gₖ. C is a regularization parameter, and b is a bias term. Given the graph Gᵢ, the bias term b can be computed by

$$b = {y}_{i} - {\sum }_{j = 1} {α}_{j} {y}_{j} K ( {G}_{i} , {G}_{j} ) \qquad ( 2 )$$

and the decision function is defined as:

$$f ( G ) = {\sum }_{i = 1}^{N} {α}_{i} {y}_{i} K ( {G}_{i} , G ) + b \qquad ( 3 )$$

Embodiments may perform a binary graph classification on graph EHR. Given success and failure cases with their associated label (gᵢ yᵢ), we want to learn a classifier such that f(gᵢ)=yᵢ where yᵢ∈{0, 1} to predict the success or failure outcome yᵢ of the given prescription in gᵢ. Embodiments may handle this problem via a kernelized support vector machine (Kernel-SVM) with a graph kernel as described below.

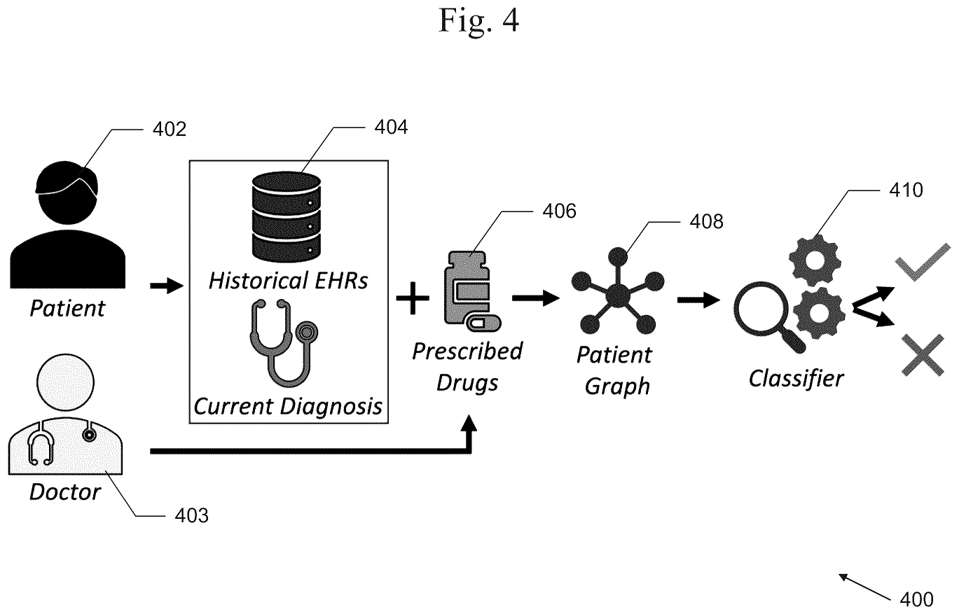

An exemplary embodiment of a predictive framework 400 directed to predicting effectiveness of a prescribed drug is shown in FIG. 4. Predictive framework 400 may include information relating to a patient 402, such as historical EHRs, information from a doctor 403, the current diagnosis 404 and the prescribed drug 406, which may be used to generate a patient graph 408, as described above, and then to generate a classifier model 410, which may be used to predict effectiveness of the prescribed drug.

In embodiments, an EHR-based graph kernel may include a Temporal Topological Kernel. To provide an effective treatment, considering the temporal relationships between medical events may be necessary. Embodiments may utilize a temporal topological kernel Kₜₚ. Specifically, input graphs may be transformed to shortest path graphs and define the kernel function as follows:

Definition 2 (Temporal topological kernel). Let g₁=(V₁, E₁) and g₂=(V₂, E₂) denote the shortest path graph of P_{g1} and P_{g2} by using the transformation discussed above, we define Temporal topological kernel Kₜₚ as:

$${K}_{tp} ( {g}_{1} , {g}_{2} ) = {\sum }_{{e}_{1} ∈ {E}_{1} , {e}_{2} ∈ {E}_{2}} {K}_{ts} ( {e}_{1} , {e}_{2} ) \qquad ( 4 )$$

where Kₜₛ is a temporal substructure kernel defined on edges e₁=(u₁, v₁) and e₂=(u₂, v₂) which calculates temporal similarity on substructures that connect to nodes in e₁, e₂.

The intuition of Kₜₚ is based on calculating the similarity among temporal ordering on substructures (e.g., node neighborhoods) by Kₜₛ between input graphs, recursively. If two graphs are similar, their temporal order for node neighborhood structures are similar. That is, for a given pair of nodes v₁, v₂ from two similar graphs g₁, g₂, the time difference from other nodes uᵢ, uⱼ in gᵢ, g₂ to v₁, v₂ where uᵢ, uⱼ lie in the subtrees that connect to v₁, v₂ must be similar.

Definition 3 (Temporal substructure kernel). Given a pair of edge e₁=(u₁, v₁), e₂=(u₂, v₂), their associated edge weight function w₁, w₂ of g₁, g₂, and set of neighbor nodes N₁, N₂ of u₁, u₂, we define temporal substructure kernel Kₜₛ as:

$${K}_{t s} ( {e}_{1} , {e}_{2} ) = {\sum }_{{{e}_{i} = ( {n}_{i} , {u}_{i} ) ∈ {E}_{1} , {n}_{i} ∈ {N}_{1}}_{{e}_{j} = ( {n}_{j} , {u}_{2} ) ∈ {E}_{2} , {n}_{j} ∈ {N}_{2}}} {K}_{ts} ( {e}_{i} , {e}_{j} ) \qquad ( 5 )$$

$${K}_{time} ( {w}_{1} ( {e}_{1} ) , {w}_{2} ( {e}_{2} ) ) \times {K}_{node} ( {u}_{1} , {u}_{2} ) \times {K}_{node} ( {v}_{1} , {v}_{2} )$$

and base case definition for the recursion part in Equation 5 when u₁ or u₂ is the root node:

$${K}_{t s} ( {e}_{1} , {e}_{2} ) = {K}_{time} ( {w}_{1} ( {e}_{1} ) , {w}_{2} ( {e}_{2} ) ) \times {K}_{node} ( {u}_{1} , {u}_{2} ) \times {K}_{node} ( {v}_{1} , {v}_{2} ) , \qquad ( 6 )$$

where Kₜᵢₘₑ is defined as:

$${K}_{time} ( {w}_{1} ( {e}_{1} ) , {w}_{2} ( {e}_{2} ) ) = {e}^{- 1 \times \vert {w}_{1} ( {e}_{1} ) - {w}_{2} ( {e}_{2} ) \vert} , \qquad ( 7 )$$

and K_{node} is defined as:

$${K}_{node} ( {u}_{1} , {u}_{2} ) = \{ \begin{matrix}1 , & i f label ( {u}_{1} ) = label ( {u}_{2} ) \\ 0 , & otherwise\end{matrix} \qquad ( 8 )$$

To show Kₜₛ is a valid kernel, it must be shown that it is positive definite.

Proof. K_{node} is a Dirac delta function which is proven to be positive definite. Kₜᵢₘₑ is positive definite since the transformation of an exponential function is positive definite. It is known that positive definiteness is closed under positive scalar linear combination and multiplication on positive definite kernels, and it holds in the base case definition in Equation 6. As a result, Kₜₛ is positive definite, and Kₜₚ is therefore positive definite.

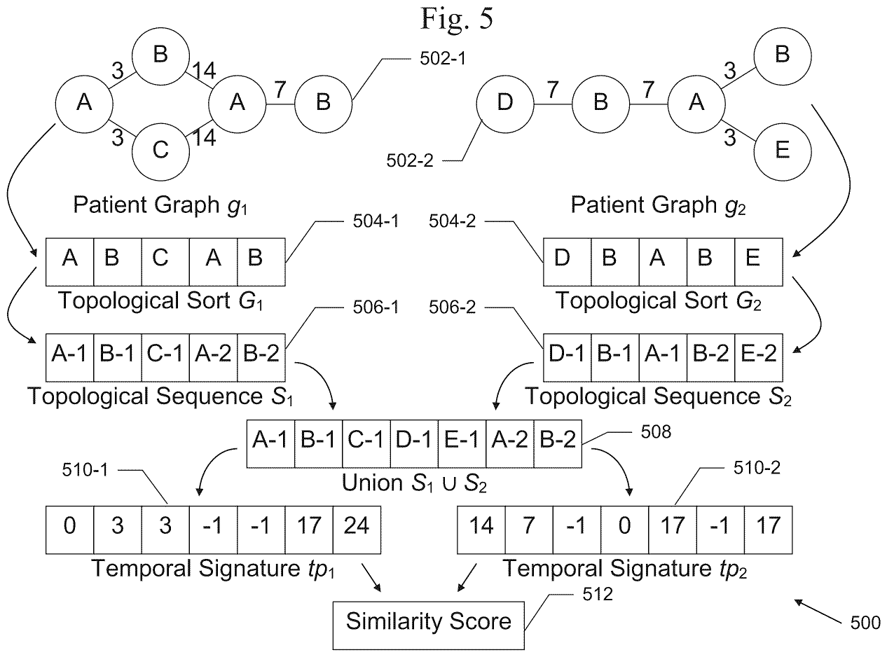

Embodiments may, for a given pair of graph input g₁, g₂, calculate their kernel value via a kernel function. In embodiments, an EHR-based graph kernel may include a Temporal proximity kernel, which requires definition of a Topological sequence and a and Temporal signature. An exemplary flow diagram of a process 500 of transferring the input from patient graphs to temporal signatures is shown in FIG. 5. As shown in FIG. 5, process 500 begins with patient graphs g₁ 502-1, g₂ 502-2. At 504-1, 504-2, a topological sort is performed on each patient graph g₁ 502-1, g₂ 502-2 to form topological sequences 506-1, 506-2.

To define a topological sequence 506-1, 506-2, let T be a topological ordering of graph G=(V, E) such that T={nᵢ|i=1, . . . , |V|}, the topological sequence S is defined as

$$S = \{ {n}_{i} . label + level \vert i = 1 , … , \vert V \vert , and {n}_{i} ∈ T \} \qquad ( 9 )$$

where + represents the string concatenation and level denotes the order of occurrence of label associated to node nᵢ in T. Namely, every node in the topological sequence has an attached number to indicate the level. The level indicates the order of occurrence of the same node label in the topological ordering.

At 508, unions of topological sequences 506-1, 506-2 are performed to generate temporal signatures 510-1, 510-2. To define a topological signature, let S₁, S₂ be topological sequences of two input graphs g₁, g₂, and S=S₁∪S₂ with the union set length m=|S|. Define the temporal signature for g₁ as tp₁={v₁₁, . . . , vₗₘ} where

$${v}_{{1}_{j}} = \{ \begin{matrix}{d}_{j} , & if S [ j ] ∈ {S}_{1} \\ - 1 , & otherwise\end{matrix} , for j = 1 , … , m$$

and define the temporal signature for g₂ as tp₂={v₂₁, . . . , v₂ₘ} where (10)

$${v}_{{2}_{j}} = \{ \begin{matrix}{d}_{j} , & if S [ j ] ∈ {S}_{2} \\ - 1 , & otherwise\end{matrix} , for j = 1 , … , m \qquad ( 11 )$$

for dⱼ denotes the total passage day from the root node to node nⱼ in its belonging patient graph. Thus, g₁ 502-1, g₂ 502-2 have been transferred into their vector representations tp₁ 510-1, tp₂ 510-2.

At 512, a similarity score may be computed. For example, a temporal proximity kernel Kₜₚ may calculate the kernel value between g₁, g₂ via temporal signature tp₁, tp₂ as:

$${K}_{tp} ( {g}_{1} , {g}_{2} ) = {e}^{- ‖ {tp}_{1} - {tp}_{2} ‖} \qquad ( 12 )$$

where ∥tp₁−tp₂∥ is the Euclidean distance between tp₁, tp₂.

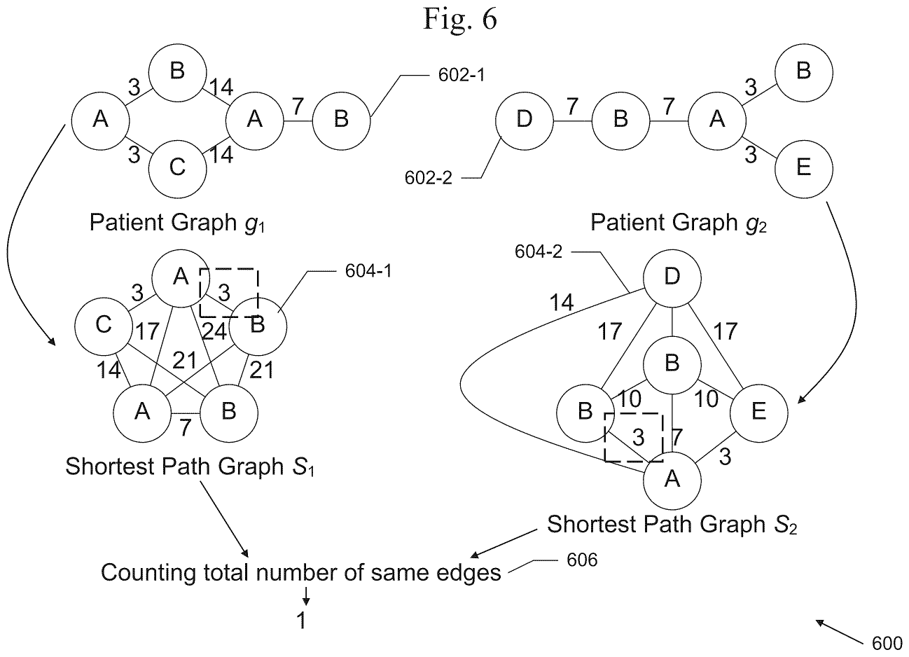

An exemplary flow diagram of a process 600 of transferring the input from patient graphs to a shortest path kernel is shown in FIG. 6. As shown in FIG. 6, process 600 begins with patient graphs g₁ 602-1, g₂ 602-2. At 604-1, 604-2, a shortest path graphs are generated from each patient graph g₁ 602-1, g₂ 602-2. At 606, a shortest path kernel Kₛₚ calculates the edge walk similarity on the shortest path graphs for two input graphs, for example by counting the total number of edges that are the same.

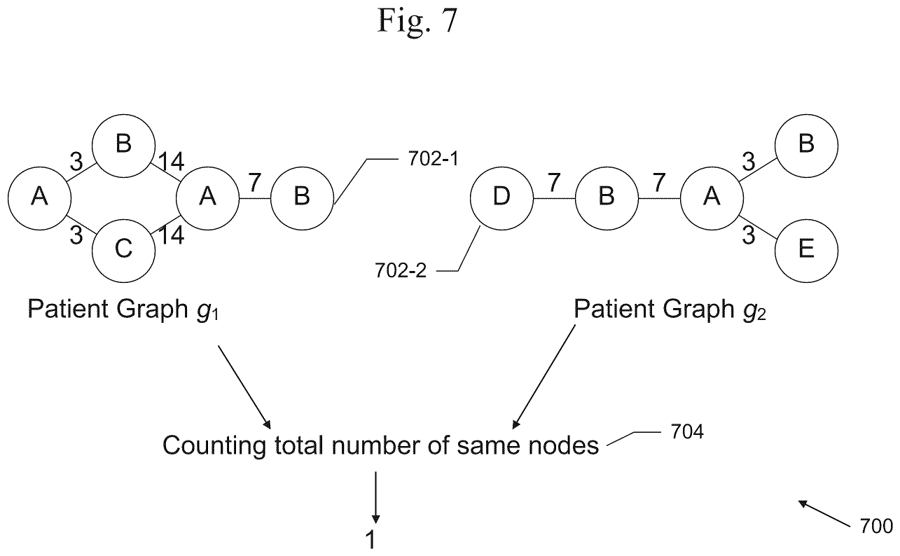

An exemplary flow diagram of a process 700 of transferring the input from patient graphs to a node kernel is shown in FIG. 7. As shown in FIG. 7, process 700 begins with patient graphs g₁ 702-1, g₂ 702-2. At 704, a node kernel K_{node} compares the node labels of two input graphs. The kernel value is the total number of same node labels:

$${K}_{node} ( {g}_{1} , {g}_{2} ) = {\sum }_{{n}_{1} ∈ {g}_{1} , {n}_{2} ∈ {g}_{1} , V} {K}_{label} ( {n}_{1} , {n}_{2} ) \qquad ( 13 )$$

where K_{label} is defined as:

$${K}_{label} ( {n}_{1} , {n}_{2} ) = \{ \begin{matrix}1 , & if label ( {n}_{1} ) = label ( {n}_{2} ) \\ 0 , & otherwise\end{matrix} \qquad ( 14 )$$

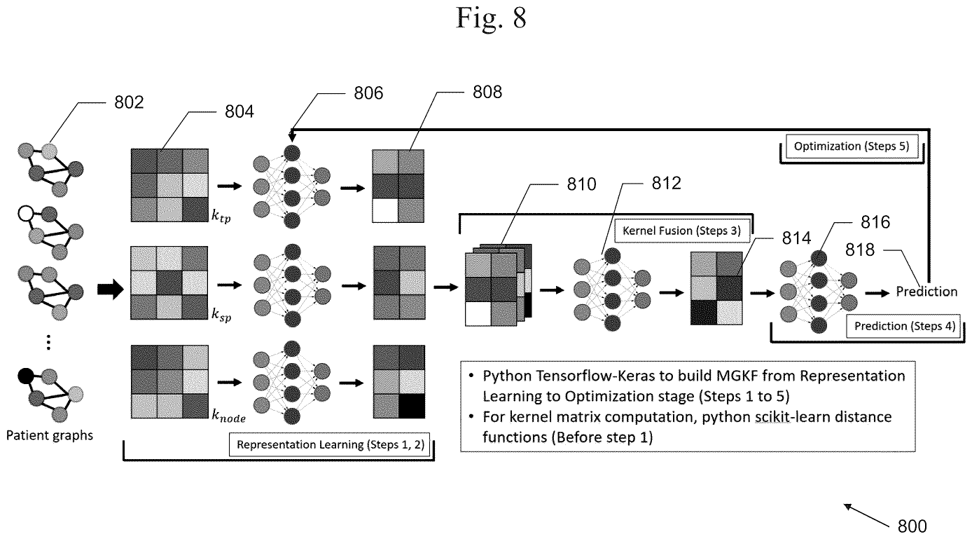

Embodiments may utilize a multiple graph kernel fusion architecture (MGKF) 800 to perform graph classification, a process of operation of which is shown in FIG. 8. At 802, a plurality of patient graphs may be received/input. At 804, a plurality of kernel gram matrices may be generated. To capture multi-view characteristics on patient graphs, two additional kernels may be used—the shortest path kernel and the node kernel, in conjunction with the temporal proximity kernel. The best combination of these kernels may be found in an end-to-end manner. Specifically, temporal proximity kernel Kₜₚ focuses on temporal similarity between substructure such as node ordering and their time difference, shortest path kernel Kₛₚ aims to capture similarity in overall connection, and node kernel K_{node} offers a balance between local and global similarity by comparing all node labels between two patient graphs to achieve best accuracy as well as prevent overfitting from noise collaboratively by kernels.

Given kernel gram matrices on all pair of n graphs for each kernel type Kₜ∈R^{n×n} where Kₜ gᵢ, gⱼ=kₜ (gᵢ, gⱼ) and t∈{tp, sp, node}, at 806, a Multi-layer perceptron (MLP) may be used to preform representation learning to generate the corresponding kernel embedding 808 representation g_{emb}_{ₜ}∈R^{n×m} where m<<n. In this case, each row i in Kₜ represents a high-dimensional feature vector with each dimension being a kernel value (e.g., similarity score) between its associated graph gᵢ and all other graphs, and its kernel embedding g_{emb}_{ₜ} can be treated as a dimension reduction by using traditional kernel approximation techniques to generate low dimensional features for gᵢ such that efficient linear classifier can be used directly. gᵢ_{ₜ}∈Rⁿ is converted to g_{emb}_{ₜ}∈R^{m} under kernel type t as follows:

$${g}_{e m {b}_{t}} = R e L U ( {W}_{t} {g}_{{i}_{t}} + {b}_{t} ) \qquad ( 15 )$$

by using the kernel embedding weight matrix Wₜ∈R^{m×n} and the bias vector bₜ∈R^{m} where n is the number of input graphs, and m is the dimension for the embedding space. The rectified linear unit (ReLU) activation is defined as ReLU (val)=max(val, 0). For deep architecture, the layer/may be computed with its previous layer l−1 with related parameters Wₜₗ and bₜₗ within layer by using the same way that we compute the embedding for input kernel gram matrix such as:

$${g}_{e m {b}_{t}} = R e L U ( {W}_{{t}_{i}} {g}_{e m {b}_{{t}_{l - 1}}} + {b}_{{t}_{l}} )$$

At 810, to combine three kernels, their embeddings 808 from the last layer may be averaged and at 812 may be fused to generate the kernel fusion g_{emb}_{_{F}} 814 using another dense layer with ReLU activation that learns the kernel fusion g_{emb}_{_{F}}∈R^{f}:

$${g}_{e m {b}_{s u m}} = {\sum }_{t ∈ \{ tp , sp , n o d e \}} {g}_{e m {b}_{{t}_{l a s t}}} \qquad ( 17 )$$

$${g}_{e m {b}_{a v g}} = \frac{{g}_{e m {b}_{s u m}}}{3}$$

$${g}_{e m {b}_{F}} = R e L U ( {W}_{F} {g}_{e m {b}_{a v g}} + {b}_{F} )$$

in which W_{F}∈R^{f×q} is the fusion weight matrix with fusion embedding dimension f and the bias vector b_{F}∈R^{f} assuming the last embedding layer dimension is q.

Further, at 816, the prediction 818 of the label of success or failure for g_{emb}_{_{F}} may be produced by using a Sigmoid layer defined as:

$$\hat{y} = Sigmoid ( {W}_{p} {g}_{e m {b}_{F}} + {b}_{p} ) \qquad ( 18 )$$

where Wₚ∈R^{1×f} and bₚ∈R are trainable weights used to generate class label ŷ∈{0, 1}. A binary cross-entropy loss function may be used to optimize the best embedding under the fusion setting to learn all kernel embedding weight matrices.

In embodiments, each row (for example, each patient) depicts a high dimensional feature vector with each dimension corresponding a kernel value to a specific training example. Since the kernel value can be treated as a similarity measurement, the concept in similarity-based classification may be used, in which class labels are inferred by a set of most similar training examples, and the top k most similar patients may be consulted to get prediction insights based on the nature that features with higher weight contribute more to the result in a linear classifier. Kernel embedding, reducing input dimension, for each kernel type facilitates similarity measurement refinement, reducing the number of training examples used to infer. Similar patients with allied graph similarity may be grouped into one coordinate (e.g., dimension) in the embedding space.

Since kernel embedding space is trained in an end-to-end manner through ReLU operation in Equation 16, which achieves the interpretability, a set of candidates may be selected that contribute most to the prediction, via top k value coordinates in the embedding space. The selected ones under different kernel type can be interpreted as multi-view representative cases (such as time propagation or disease connection) in case-based learning. In practice, patient g_{emb}_{ₜ} may be sorted in kernel embedding space, and the top k coordinates may be selected. The top k′ training examples for the i-th coordinate in top k coordinates may be selected. All sorts are in a reverse order:

$$argsort ( {g}_{e m {b}_{t}} ) [ 1 : k ]$$

$$argsort ( {W}_{t} [ i , : ] ) [ 1 : {k}^{′} ]$$

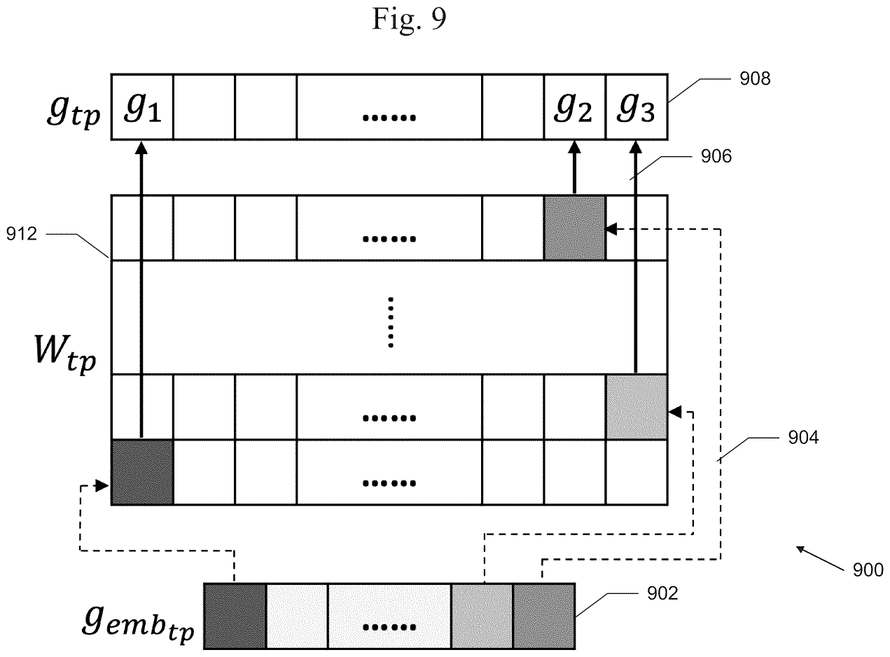

An exemplary process 900 of interpretation is shown in FIG. 9. Given an embedded patient vector g_{emb}_{ₜₚ} 902, at 904, it may be sorted in a descending order and, for example, the top three value dimensions may be selected. At 906, the training examples in gₜₚ 908, which contribute most may be found, through weight matrix Wₜₚ 912.

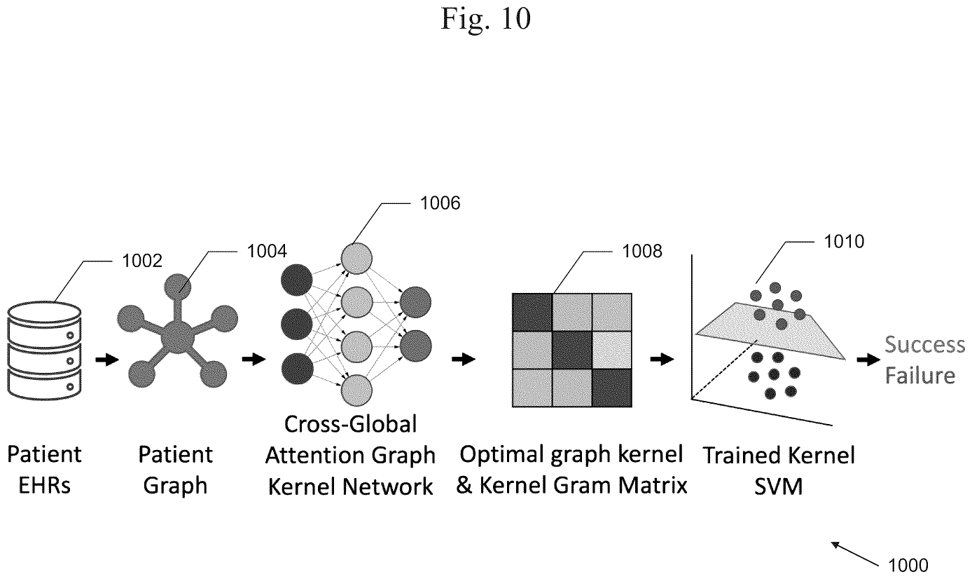

An exemplary embodiment of a predictive framework 1000 directed to predicting effectiveness of a course of treatment for a chronic condition is shown in FIG. 10. Predictive framework 1000 may include information relating to a patient 1002, such as patient EHRs, which may be used to generate a patient graph 1004, as described above. Patient graph 1004 may be used to generate a Cross-Global Attention Graph Kernel Network 1006, which may be used to generate an optimal graph kernel and Kernel Gram Matrix 1008 which may be used to generate a trained Kernel SVM 1010, which may be used to predict effectiveness of the course of treatment.

Embodiments may formulate the prediction task as a binary graph classification on graph-based EHRs 1004 using a kernel SVM 1010. Such embodiments may learn a graph kernel. Given a set of success and failure case patient graphs G, a deep neural network may learn an optimal graph kernel k. Then, the prediction for success or failure is performed by a kernel SVM 1010 using a kernel gram matrix K 1008 such that Kᵢⱼ=k(Gᵢ, Gⱼ) where Gᵢ, Gⱼ∈G. For an incoming patient, a patient graph Gₚ 1004 may be created based on the concatenation of patient's medical history, current diagnosis, and treatment plan. Then, the kernel value between Gp and all training examples Gi∈G may be determined, and prediction may be performed through a kernel SVM 1010, as shown in FIG. 10.

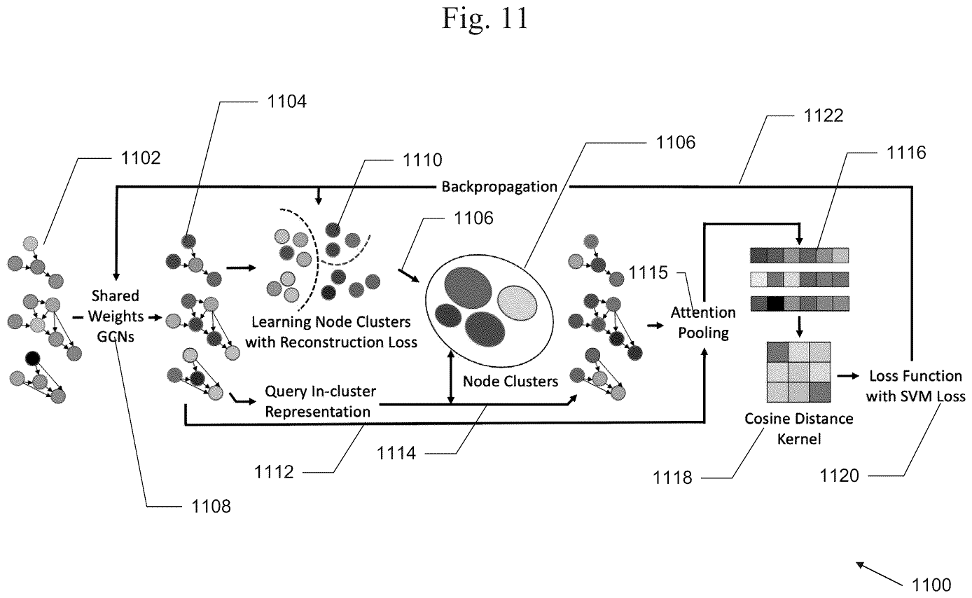

In embodiments, a Cross-Global Attention Graph Kernel Network 1100 may learn an end-to-end deep graph kernel on a batch of graphs, as shown in FIG. 11. At 1102, a plurality of patient graphs may be received/input. The node level embedding 1104 and node clusters 1106 may be determined first by, at 1108 determining shared weight Graph Convolutional Networks (GCNs) to form node level embedding 1104, and at 1110, determining learning node clusters with reconstruction loss to form node clusters 1106. The graph level embedding may be derived 1112 from node matching 1114 based attention pooling 1115. The loss 1120 may be calculated 1116 by the resulting distance and kernel matrix 1118 and backpropagation 1122 may be performed to update all model parameters.

As shown, this may be accomplished through cross-global attention node matching without an explicit pairwise similarity computation. Given a batch B of input graphs G₁, . . . , G_{|B|} with batch size |B|, their nodes may be embedded into a lower dimensional space, where node structures and attribute information are encoded in dense vectors. A graph level embedding may then be produced by a graph pooling operation on node level embedding via cross-global attention node matching. The batchwise cosine distance may be calculated and a kernel gram matrix may be generated on the entire batch of resulting graph embedding. Finally, the network loss may be computed with contrastive loss, kernel alignment, and SVM primal objective.

Embodiments may perform Graph Embedding using Graph Convolutional Networks. Graph Convolutional Networks (GCN) may perform 1-hop neighbor feature aggregation for each node in a graph. The resulting graph embedding is permutation invariant when a pooling operation is properly chosen. Given an n number of nodes patient graph G with node attribute one-hot vector matrix X∈R^{n×c}, where c denotes the total number of medical codes in EHRs, and a weighted adjacency matrix A∈R^{n×n}, GCN may be used to generate a node level embedding H∈R^{n×d} with embedding size d∈R as follows:

$$H = f ( {\tilde{D}}^{- 1} \tilde{A} XW )$$

where \tilde{D} is the diagonal node degree matrix of Ã defined with \tilde{D}=ΣjÃᵢⱼ, Ã=A+I is the adjacency matrix with self-loops added, W∈R^{c×d} is a trainable weight matrix, and f is a non-linear activation function such as ReLU (x)=max (0, x). The embedding H can be an input to another GCN, creating stacked multiple graph convolution layers:

$${H}^{k + 1} = f ( {\tilde{D}}^{- 1} \tilde{A} {H}^{k} {W}^{k} ) , {H}^{0} = X$$

where H^{k} is the node embedding after the k^{th} GCN operation, and W^{k} is the trainable weight associated with the k^{th} GCN layer. The resulting node embedding H^{k}+1 contains k-hop neighborhood) structure information aggregated by graph convolution layers.

Embodiments may perform graph embedding using higher-order graph information. To capture longer distance nodes and their hierarchical multi-hop neighborhood information, t multiple GCN layers may be stacked and all layer's outputs H^{1:}^{ₜ}=[H¹, . . . , H^{t}] concatenated, where H^{1:}^{ₜ}∈R^{n×(t×d)}. The concatenated node embedding might be very large and could potentially cause a memory issue for subsequent operations. To mitigate such drawbacks, we perform a non-linear transformation on H^{1:}^{ₜ} by a trainable weight W_{concat}∈R^{(t×d)×d} and a ReLU activation function as follows: (23)

$${H}_{final} = ReLU ( {H}^{1 : t} {W}_{concat} )$$

To produce the graph level embedding, instead of using another type of pooling operation, embodiments may use cross-global attention node matching and its derived attention based pooling.

Cross-Global Attention Node Matching between graphs may be computed via a pairwise node similarity measurement. This optimizes a distance metric-based or KL-divergence loss on the graph pairs or triplets necessitating vast training pairs or triplets to capture the entire global characteristics. One way to avoid explicit pair or triplet generation utilizes efficient batch-wise learning via optimizing classification loss. However, pairwise node matching in a batch-wise setting is problematic due to graph size variability. To address this issue, one may use a batch-wise attention-based node matching scheme, also known as cross-global attention node matching. The matching scheme may learn a set of global node clusters and may compute the attention weight between each node and the representation associated with its membership cluster. The pooling operation based on its attention score to global cluster may perform a weighted sum on nodes to derive a single graph embedding.

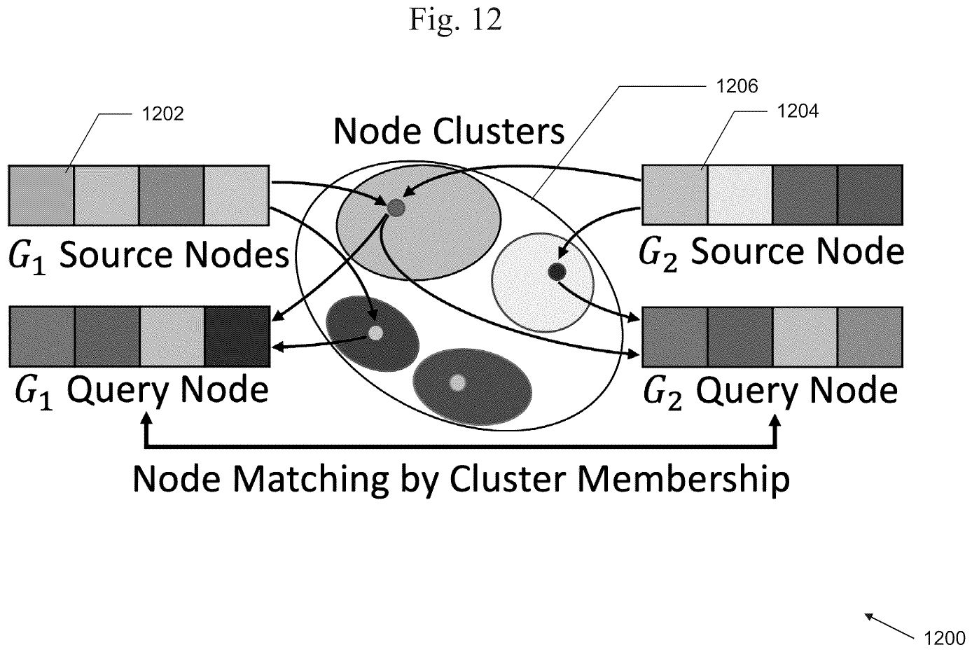

Given node embedding H_{final}∈R^{n×d} from the last GCN layer and transformation after concatenation in Equation 23, define M∈R^{s×d} as a trainable global node cluster matrix with s clusters and d dimension features sized to provide an overall representation of its membership nodes. Here, define membership assignment A∈R^{n×s} for H_{final} and as follows:

$$A = Sparsemax ( R e LU ( {H}_{final} {M}^{T} ) ) \qquad ( 24 )$$

where Sparsemax is a sparse version Softmax, that outputs sparse probabilities. It can be treated as a sparse soft cluster assignment. A may be interpreted as a cluster membership identity with s dimension feature representation. Further define the query of nodes' representation in their belonging membership cluster:

$$Q = Tanh ( AM ) \qquad ( 25 )$$

where Q∈R^{n×d} denotes a queried representation for each node in H_{final} from their belonging membership cluster. As shown in FIG. 12, matching can be treated as retrieving cluster identity from global node clusters, and similar nodes are assigned to a similar or even the same cluster membership identity. To construct a better cluster, we add an auxiliary loss by minimizing the reconstruction error L_{recon}=∥H_{final}−Q∥ F, which is similar to Non-negative Matrix Factorization (NMF) clustering.

Embodiments may utilize Pooling with Attention-based Node Matching. The intuition of pairwise node matching is to assign higher attention-weight to those similar nodes. In other words, matching occurs when two nodes are highly similar, closer to each other than to other possible targets. Following this idea, it is observed that two nodes are matched if they have similar or even identical cluster membership. The higher the similar membership identity, the higher the degree of node matching. In addition, a cluster is constructed by minimizing the reconstruction error between the original node H_{final} and the query representation Q. A node with high reconstruction error means no specific cluster assignment and further lowers the chance to match other nodes. This can be measured by using similarity metrics (e.g., cosine similarity) between H_{final} and Q. Based on these observations, cross-global attention node matching pooling may be designed, wherein a node similar to the representation in its cluster membership should receive higher attention weight, as follows:

$$a = Softmax ( Sim ( {H}_{final} , Q ) ) \qquad ( 26 )$$

$${G}_{emb} = {\sum }_{i = 1}^{n} {a}_{i} {H}_{{final}_{i}}$$

where α∈Rⁿ is the attention weight for each node, Softmax is applied to generate importance among nodes by using Sim, a similarity metric (e.g., cosine similarity), and the resulting pooling G_{emb} is the weighted sum of node embeddings that compress higher order structure and node matching information from other graphs.

Matching and cluster assignment membership is illustrated in FIG. 12, which shows a predictive framework 1200. Each node in G₁ 1202, G₂ 1204 may map to a cluster 1206. Their cluster membership assignments may generate their query, which is their representation in terms of belonging to a cluster. Such an assignment may be seen as a soft label of cluster membership identity. Similar query means similar cluster membership identity, inducing possible matching.

Graph Kernel. Given a graph pair with their graph level embeddings G_{emb}_{₁}, G_{emb}_{₂}, the graph kernel may be defined as follows:

$${Dist}_{C} ( {G}_{e m {b}_{1}} , {G}_{e m {b}_{2}} ) = 1 - \frac{{G}_{e m {b}_{1}} \cdot {G}_{e m {b}_{2}}}{‖ {G}_{e m {b}_{1}} ‖ ‖ {G}_{e m {b}_{2}} ‖} \qquad ( 27 )$$

$${Dist}_{E} ( {G}_{e m {b}_{1}} , {G}_{e m {b}_{2}} ) = {‖ {G}_{e m {b}_{1}} - {G}_{e m {b}_{2}} ‖}_{2}$$

$$K ( {G}_{e m {b}_{1}} , {G}_{e m {b}_{2}} ) = exp ( - {Dist ( {G}_{e m {b}_{1}} - {G}_{e m {b}_{2}} )}^{2} )$$

where Dist_{C} is a cosine distance and Dist_{ε} is the Euclidean distance. Dist can be either Dist_{C} or Dist_{ε}. The resulting kernel function is positive definite since exp(−x) is still positive definite for any non-negative real number x. Cosine distance enjoys benefits in more complex data representations. Euclidean distance considers vector magnitude (such as the norm) during measurement which is not sufficiently sensitive to highly variant features such as long-term disease progressions. Moreover, cosine distance can measure objects on manifolds with nonzero curvature such as spheres or hyperbolic surfaces. In general, Euclidean distance can only be applied to local problems which may not be sufficient to express complex feature characteristics. The resulting cosine guided kernel is more expressive, and thus, capable of performing implicit high dimensional mapping. Note that the use of other distance functions that support a positive definitive kernel is likewise within the scope of this disclosure,

Given a batch B of input graphs and their class labels y∈R^{|B|×1} where yᵢ∈{1, 0}, we get their graph level embeddings for the entire batch via shared weight GCN with cross-global node matching pooling. Then, we calculate their batch-wise distance matrix D^{∈R|B|×|B|} and batch-wise kernel gram matrix K∈R^{|B|×|B|}. The model can be trained by mini-batch Stochastic Gradient Descent (SGD) without training pair and triplet generation. To learn an optimal graph embedding, which results in an optimal graph kernel, we optimize it by contrastive loss with a margin threshold λ>0 and kernel alignment loss:

$${ℒ}_{contrastive} = \frac{1}{\vert B \vert} {\sum }_{i , j ∈ B} ( 1 - {Y}_{ij} ) {max ( 0 , λ - {D}_{ij} )}^{2} + {Y}_{ij} {D}_{ij} \qquad ( 28 )$$

and kernel alignment loss:

$${ℒ}_{alignment} = \frac{1}{\vert B \vert} \sqrt{2 - 2 ( {〈 K , Y 〉}_{F} / \sqrt{{〈 K , K 〉}_{F} {〈 Y , Y 〉}_{F}}} \qquad ( 29 )$$

where [symbol] ⋅, ⋅ [symbol] F denotes the Frobenius inner product, K is a batch-wise kernel gram matrix, and Y E R^{|B|×|B|} where Yᵢⱼ=1 if yᵢ=yⱼ else Yᵢⱼ=0. A good distance-metric may induce a good kernel function and vice versa. So, the graph kernel may be learned jointly through optimal cosine distance between graphs via contrastive loss with an optimal graph kernel through kernel alignment loss:

$${ℒ}_{kernel} = {ℒ}_{contrastive} + {ℒ}_{alignment} \qquad ( 30 )$$

To align a learned embedding, distance, and kernel to the classification loss in end-to-end training, the SVM primal objective may be incorporated with a squared hinge loss function into the objective:

$${ℒ}_{SVM} = C {\sum }_{i , j ∈ B} {β}_{i} {β}_{j} {K}_{ij} + {\sum }_{i} {max ( 0 , 1 - {y}_{i} {\sum }_{j ∈ B} {K}_{ij} {β}_{j} )}^{2} \qquad ( 31 )$$

where C>=0 is a user defined inverse regularization constant and β∈R^{|B|×1} is a trainable coefficient weight vector. The following is the final model optimization problem formulation:

$$\begin{matrix}min \\ θ , β\end{matrix} \begin{matrix}{ℒ}_{kernel} \\ θ\end{matrix} + \begin{matrix}{ℒ}_{recon} \\ θ\end{matrix} + \begin{matrix}{ℒ}_{SVM} \\ β\end{matrix} \qquad ( 32 )$$

where θ denotes a set of all trainable variables in graph embedding and β is a trainable coefficient weight vector for SVM. Since the training is done by mini-batch SGD, the SVM objective is only meaningful for a given batch. Namely, gradient for β in SVM are only relevant for the current batch update as the SVM objective is dependent on the input kernel gram matrix. When training proceeds to the next batch, the kernel gram matrix is different, and the optimized β is inconsistent with the last batch status. To resolve this inconsistent weight update problem, treat SVM as a light-weight auxiliary objective (e.g., regularization), encouraging the model to learn an effective graph kernel. In this case, first perform a forward pass through graph kernel network, then train the SVM by feeding in the kernel gram matrix from the forward pass output until convergence. The positive definiteness of the kernel function guarantees SVM convergence. Once the SVM is trained, treat β as a model constant, and [symbol] _{SVM} now acts as a regular loss function. The gradient of θ can be computed through [symbol] ₖₑᵣₙₑₗ, [symbol] _{recon}, and [symbol] _{SVM}, and the model can perform backpropagation to update θ.

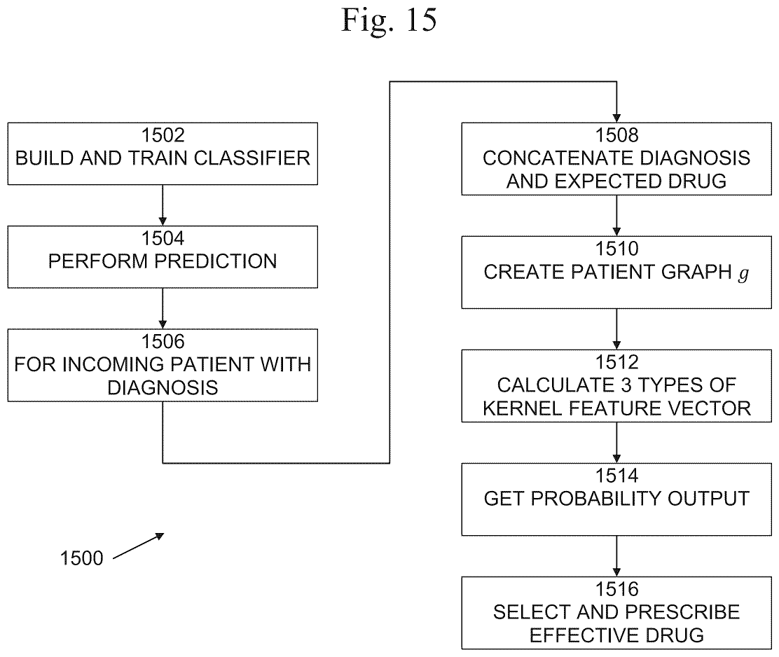

An exemplary process 1500 for predicting the outcome of a drug prescription is shown in FIG. 15. Process 1500 begins with 1502, in which a classifier with an MGKF framework may be built and trained for each type of disease. Typically, training may be performed using only patients with that type of disease to train the MGKF. At 1504, to complete the training, the trained classifiers may be used with an MGKF framework to perform prediction for each type of disease. At 1506, for an incoming patient with a diagnosis, at 1508 the diagnosis and the expected drug prescription may be concatenated to the patient's medical history (if any). At 1510, a patient graph g may be created. At 1512, three types of kernel feature vectors, for a Temporal Proximity Kernel, a Shortest Path Kernel, and a Node Kernel, may be calculated between g and all training examples. At 1514, a probability output may be obtained using the MGKF with same disease diagnosis type. For example, a probability output >0.5 may mean possible failure. At 1516, a drug or treatment that is likely to be effective, based on the probability output for that drug or treatment may be selected and prescribed to a patient.

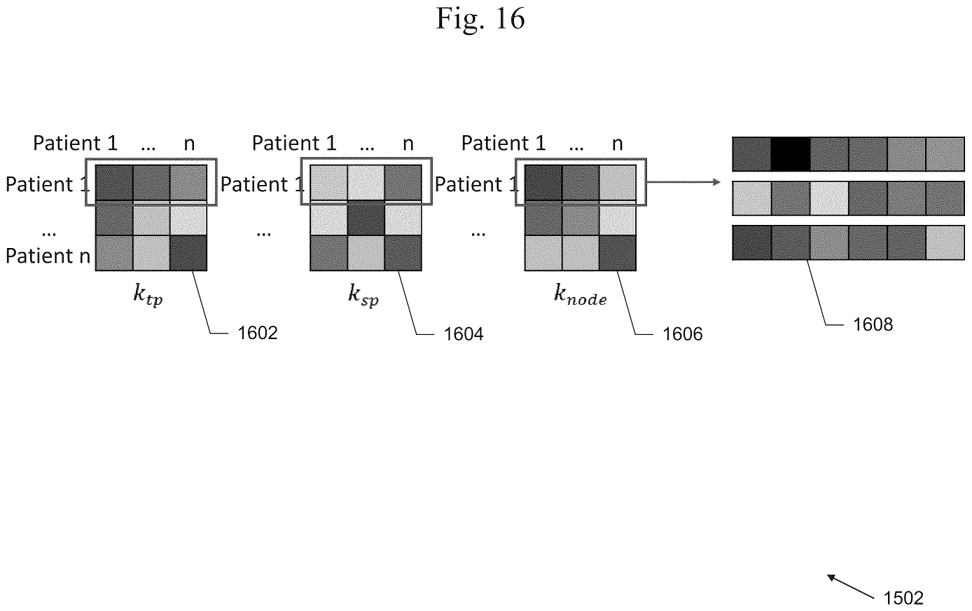

An exemplary process 1502 of training a classifier with an MGKF framework is shown in FIG. 16. Given n patient graphs under specific type of disease for example, a UTI, an n×n kernel gram matrix may be created for each of kₜₚ, kₛₚ, and k_{node}. Each row represents an n dimension feature vector for each associated patient and describes the similarity (kernel value) to all n patients. In this example, there are n patients with each patient having an n dimensional feature vector in kₜₚ, kₛₚ, and k_{node}. Each row in kₜₚ, kₛₚ, and k_{node} may be treated as a one-dimensional feature vector 1608.

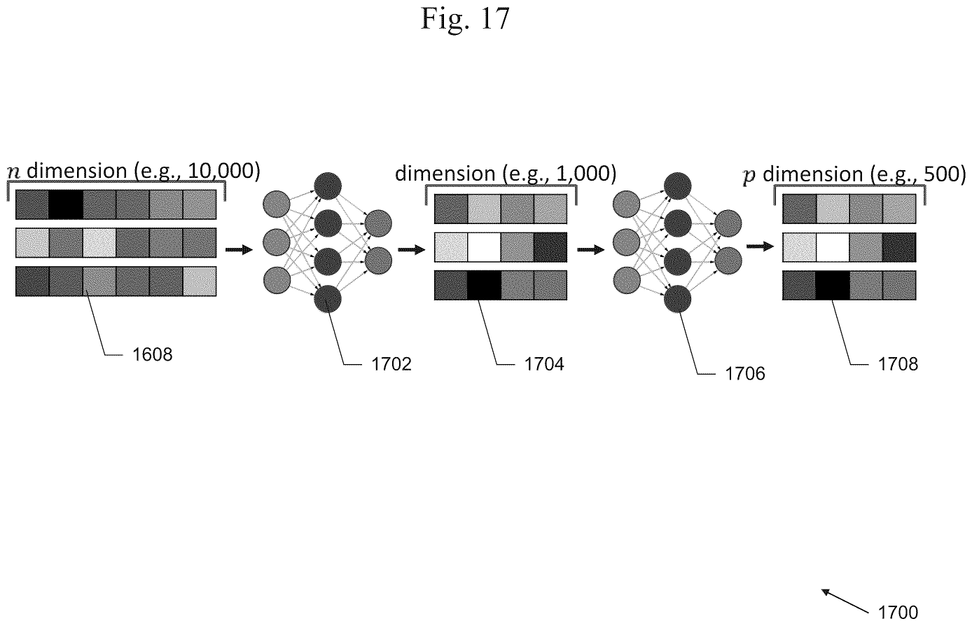

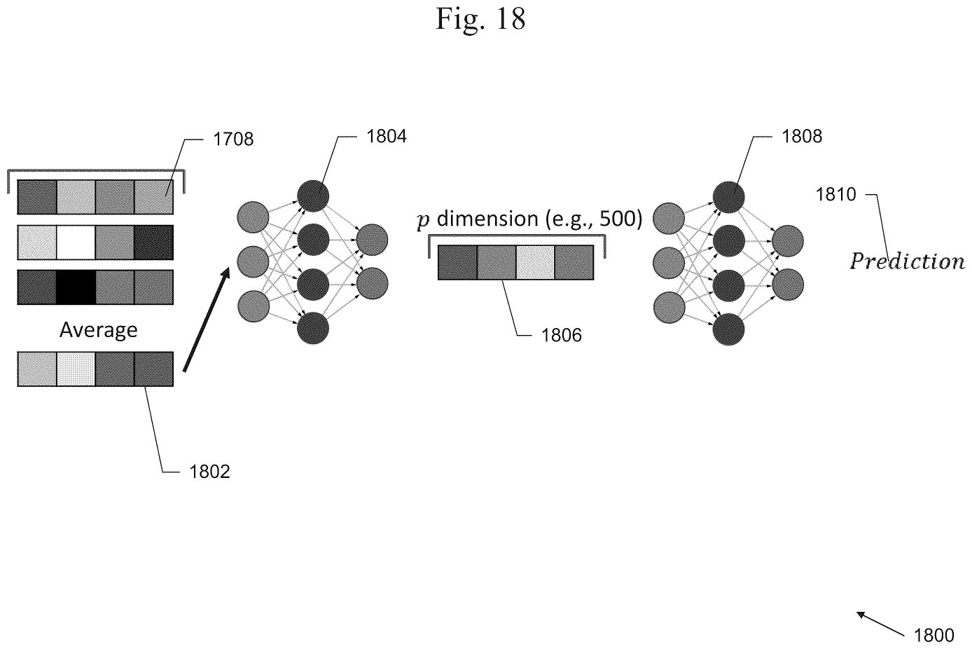

An exemplary process 1504, of using the trained classifiers with an MGKF framework to perform prediction for each type of disease is shown in FIGS. 17 and 18. The three types of feature vectors (vector of kernel values 1608) may be input into an MGKF framework to generate predictions. A portion (representation learning) 1700 of exemplary process 1504 is shown in FIG. 17. For each n dimensional feature vector 1608 from kₜₚ, kₛₚ, and k_{node}, MLP 1702 may be used to reduce the dimension from n to m. For example, at 1702 each type of feature vector may be embedded with a dimension size 10,000 to 1,000 1704 via a single layer MLP with ReLU activation (1,000 hidden size). At 1706, for each type of embedding, the dimension may, for example, be reduced from 1,000 to 500 via a 3-layer MLP with ReLU activation (hidden size for 800, 600, and 500 for each layer) to get a final representation 1708.

A portion (Kernel Fusion and Prediction) 1800 of exemplary process 1504 is shown in FIG. 18. After final representation learning (embedding) 1708 for each type of feature vectors, the feature vectors may be averaged to form a single vector and input into a single layer MLP (500 hidden size) 1804 to learn the final representation 1806. At 1808, another MLP, such as a single layer (1 hidden size) MLP with sigmoid activation may be used to output probability 1810 of likelihood for success or failure.

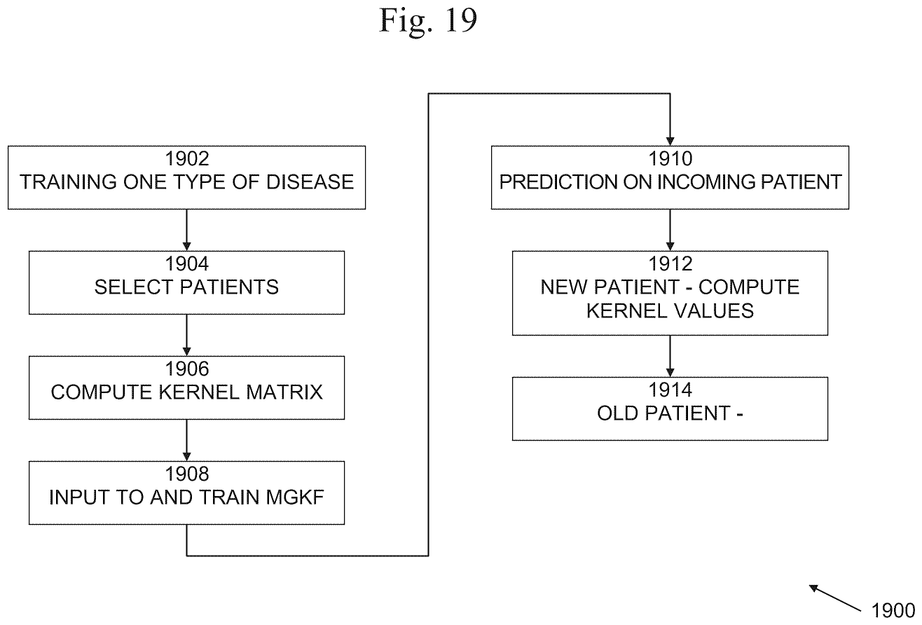

An exemplary process of predicting drug and/or treatment outcomes is shown in FIG. 19. Process 1900 begins with 1902, in which, for training for one type of disease, at 1904, n patients, for example, 10,000, may be selected under selected disease types as training examples, and their patient graphs may be created. At 1906, a pairwise kernel matrix under each type of kernel, a Temporal Proximity Kernel, a Shortest Path Kernel, and a Node Kernel, may be computed, for all patients. At 1908, the pairwise kernel matrix may be input to train the MGKF. At 1910, predictions for incoming patients may be generated. At 1912, if the patient is a new patient, that is, not included in the training examples, then the kernel values between the graph of the new patient and all training examples may be computed. If the patent is an old patient, that is, included in the training examples, then return the entire patient's belonging row in the kernel gram matrix kₜₚ, kₛₚ, and k_{node}. It may be noted that there is no need to retrain when there is a sufficiently large number of training examples.

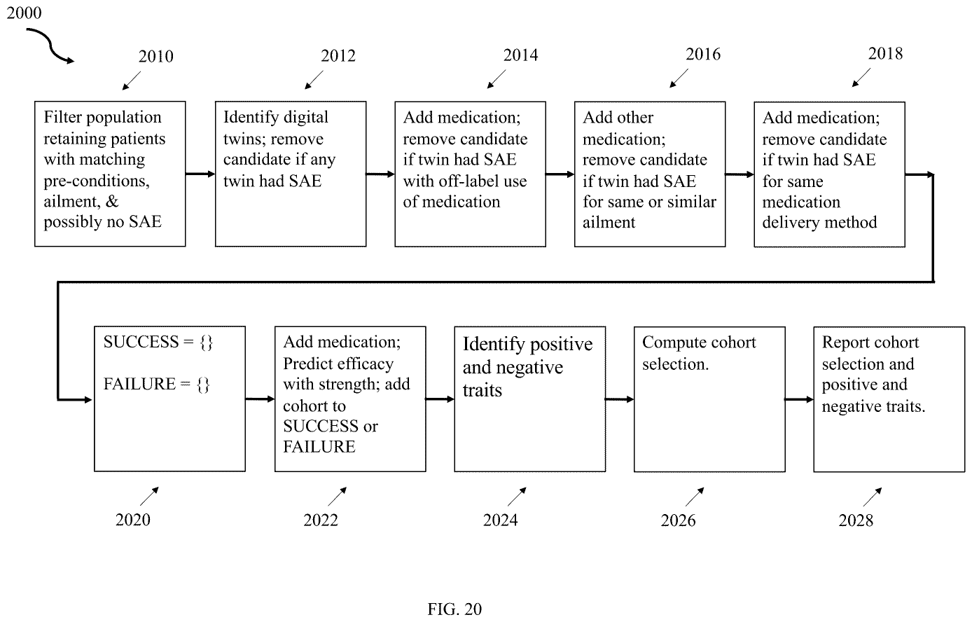

The present disclosure provides methods and systems for cohort selection. Referring now to FIGS. 20-24, methods and systems for selecting a patient cohort for a clinical trial or study are disclosed. The methods generally include identifying and mitigating likely severe adverse effects (SAE) or “too many to be acceptable”, namely excessive adverse effects (EAE), for a proposed treatment within a pool of potential candidates (e.g., cohorts (C)). The method includes identifying traits common to patients for which a proposed treatment is likely to fail, referring to these traits as negative traits (Tₙ), and identifying traits common to patients for which a proposed treatment is likely to succeed, referring to these traits as positive traits (Tₚ). The method then identifies a patient cohort based on the positive and negative traits. The method enables researchers to pre-qualify trials by selecting patients with a highest probability of success while minimizing the risk of adverse events that could potentially terminate the study. Selecting a likely-to-succeed set of patients as the study cohort can expedite approval of the treatment under consideration. Simultaneously, risks for a patient cohort that might not be suited for the medication are minimized by potentially limiting the drug target audience to an appropriate likely-to-succeed cohort, at least for initial treatment approval.

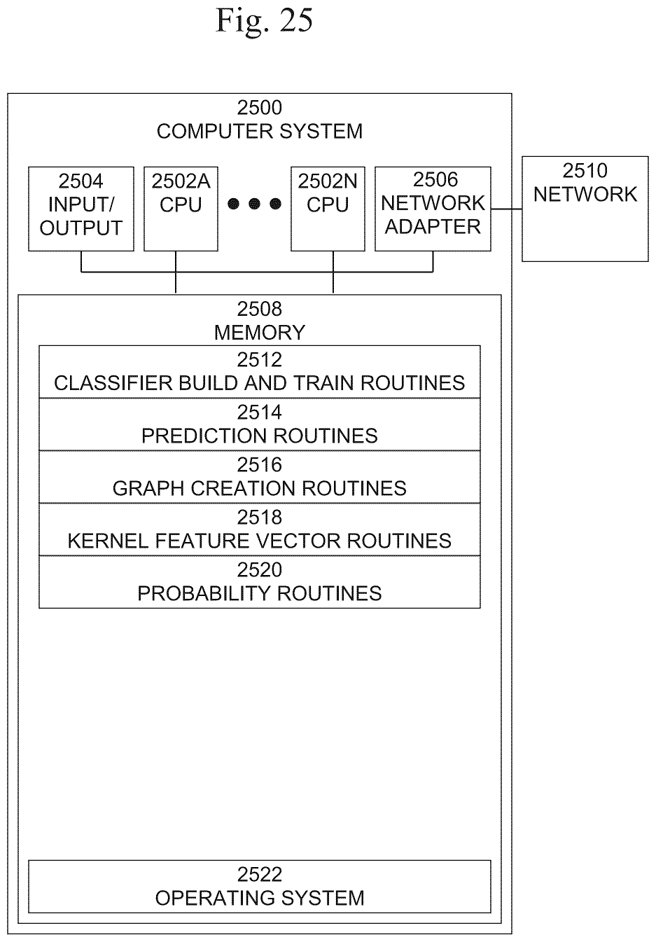

A corresponding system configured to execute the method is described in detail in FIG. 25, and generally comprises a processor and memory storing instructions for executing the cohort selection method. The system interfaces with a secondary storage device storing an electronic medical repository containing candidate medical records. The electronic medical repository includes patient medical records, treatment histories, and adverse event reports for a population of potential clinical trial participants.

FIG. 20 is a flowchart diagram showing a method 2000 for patient cohort selection according to embodiments of the disclosure. The method 2000 begins with a patient population (P) representing all potential candidates. The method 2000, via, for instance a computer processor or system, is configured to retrieve one or more medical records and construct a patient graph in which nodes represent one or more patients and attributes (e.g., demographics, comorbidities, biomarkers, and genetic markers), and edges represent one or more relationships such as shared diagnoses, durations, or treatment responses. Various subsets are defined based on adverse event history, including:
I(−): patients who exhibited adverse effects when taking the proposed medication for unrelated ailments;
O(−): patients who exhibited adverse effects when taking other medications for the same or similar ailment; and
D(−): patients who exhibited adverse effects when taking medications using a similar delivery method, such as ionizable lipid nanoparticles (LNPs).

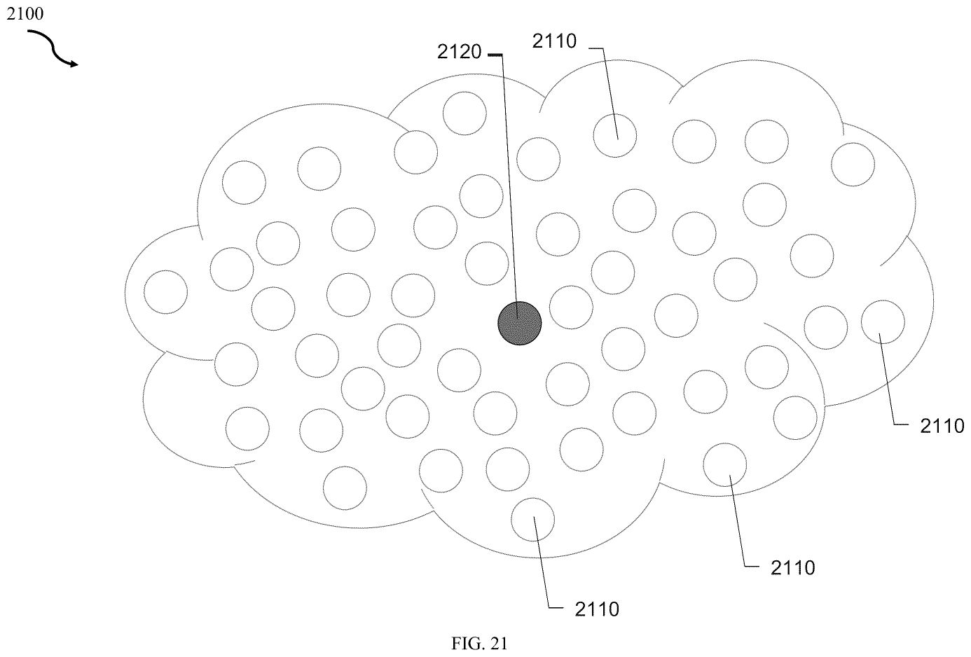

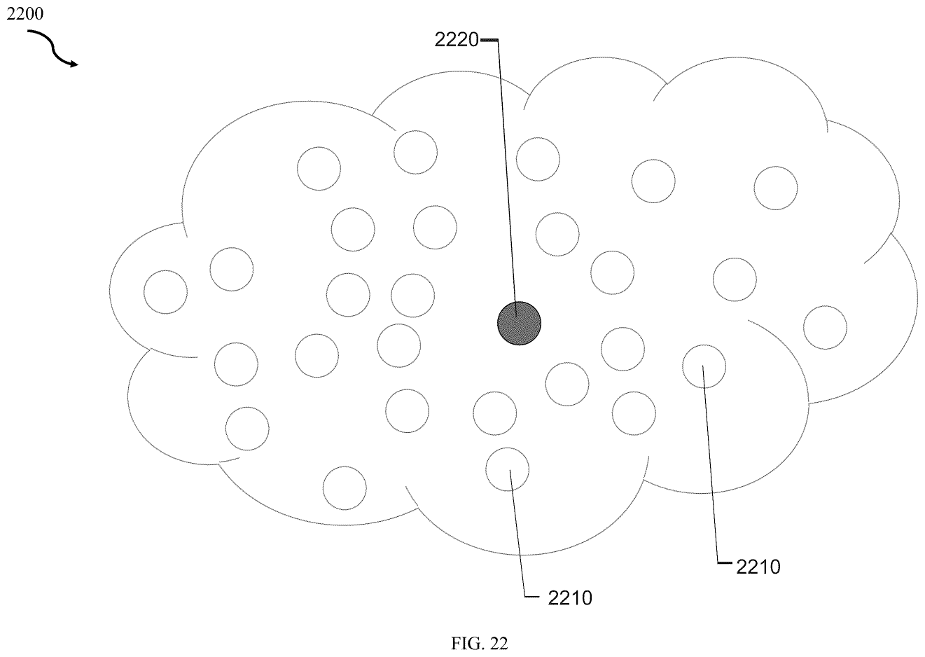

At step 2010, the method filters an initial patient population, retaining candidates with matching preconditions and/or ailments, as well as those who may not have exhibited any severe adverse events (SAE) from, for instance, similar treatments. At Step 2010, the method filters the initial patient population (P) to produce a candidate subset (C). Step 2010 includes the sub-steps of retrieving one or more patient records from an electronic medical repository, applying one or more ailment and demographic filters, and optionally excluding patients with any prior SAE history. Non-health-related filters, such as geographic proximity, conflict-of-interest factors, or prior trial participation, may also be applied. At step 2010, the method 2000 filters an initial patient population (P) based on target ailment, applies one or more demographic preconditions, optionally, excludes patients with any prior SAE history. FIG. 21 illustrates an example of a starting patient population 2100, where the unfilled circles (e.g., circles 2110) represent candidate patients. One or more unsuitable candidate patients (represented by filled circles, e.g., circle 2120) may be filtered out of the patient population. Step 2010 then produces an initial candidate subset C, which may be defined as any potential remaining potential cohort patients after filtering step 2010. FIG. 22 illustrates an example cohort set (C) 2200 after the initial filtering steps, where the unfilled circles (e.g., circles 2210) represent the remaining cohort set (C) after the first filtering step. Again, one or more unsuitable candidate patients (represented by filled circle 2220) may be filtered out of the patient population, leaving a cohort subset for further processing.

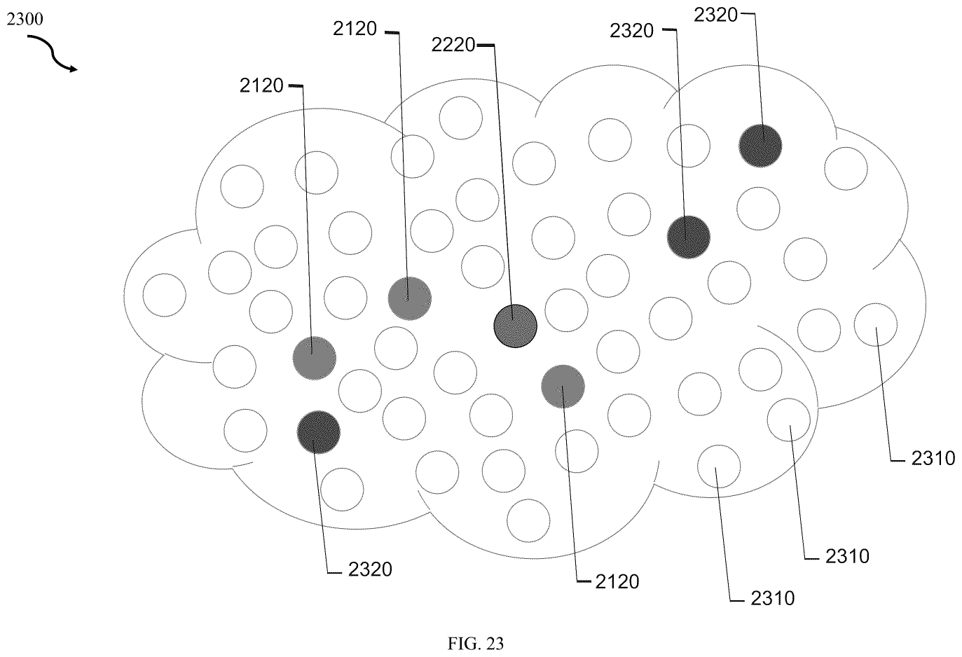

From step 2010, the method proceeds to step 2012 in which the method optionally identifies and removes one or more digital twins for each candidate patient. For each candidate patient (xᵢ∈C), the method 200 may identify a digital twin set (DT(xᵢ)) within the population (P). One or more digital twins are identified by matching demographic characteristics, medical history, genetic markers, biomarkers, and comorbidities using graph-matching algorithms. That is, for each patient (xᵢ∈C), the method is configured to identify a digital twin set (DT(xᵢ)) within the patient population (P). Patient attributes are stored in graph nodes, and similarity scoring may employ vector embeddings or distance metrics in feature space. Patients exhibiting characteristics of a digital twin in the patient population may be matched to potential candidates based on similar medical characteristics (demographics, medical history, genetic markers, biomarkers, comorbidities). Graph matching properties may be utilized with patient demographic characteristics stored in one or more patient graph nodes. FIG. 23 illustrates an example cohort set (C) 2300 after having identified one or more digital twins for a candidate patient, where the unfilled circles (e.g., circles 2310) represent the remaining cohort set (C) after the digital twins are removed. Based on this identification of one or more digital twins, one or more unsuitable candidate patients (represented by filled circle 2320) may be filtered out of the patient population, leaving a further cohort subset for further processing.

Once any identified digital twins are removed, the method 2000 moves to step 2014, in which one or more patient profiles are reviewed for a potential SAE when a proposed medication was used off-label (i.e., not used for the treatment of a condition for which the medication was originally intended and/or marketed for treatment of by the medication manufacturer). In one embodiment, EAE is treated as an SAE. At step 2014, for each candidate patient (xᵢ) the method 2000 examines the one or more digital twins (DT(xᵢ)) to verify whether any digital twin experienced an SAE or EAE when taking the proposed medication for another condition. The method 2000 then removes any candidate patient (xᵢ) from the cohort (C) if a problematic adverse effect was detected.

Once any candidate patients have been removed via step 2014, the method 2000 proceeds to step 2016, in which the use of other medications, and any adverse effects of those medications on one or more remaining candidates are reviewed for same/similar conditions. The method 2000 examines one or more digital twins for any adverse effects detected when taking medications other than proposed treatment. Treatments for a same or similar ailment as proposed in the study are examined. Candidate patients having one or more digital twins (DT(xᵢ)) that showed an SAE or EAE with alternative treatments are removed.

Upon removal of any candidate patients in step 2016, the method 2000 proceeds to step 2018, where one or more medications with the same or a similar delivery method are examined. At step 2018, the method is configured to analyze one or more digital twins for adverse effects with medications using similar delivery methods as proposed treatment. The method 2000 may analyze both one or more inert materials and/or delivery mechanisms (e.g., mRNA via ionizable lipid nanoparticles, etc.). At step 2018, candidates having one or more digital twins who experienced an SAE or EAE with similar delivery methods may be excluded.

Upon exclusion of any potential candidates failing to meet the criteria at step 2018, the cohort (C) includes one or more patients: (i) suffering from the ailment of interest, (ii) matching demographic pre-conditions, where (iii) neither the candidate patient nor any of digital twins of the candidate patient have experienced a SAE or EAE, (iv) neither the candidate patient nor any of digital twins of the candidate patient experienced an SAE or EAE when having taken the proposed medication for some other treatment, (v) neither the candidate patient nor any of digital twins of the candidate patient experienced a SAE or EAE after having taken a medication other than the one under consideration for a sufficiently similar or the same ailment now being treated, and (vi) neither the candidate patient nor any of digital twins of the candidate patient experienced an SAE or EAE when having taken a medication that relies on a similar delivery method as that considered. In any of the above steps relating to determining a level or number of SAEs or EAEs to exclude from the cohort (C), the method may adjust the exclusion threshold as desired. For instance, a threshold exclusion level may be raised or lowered based on a category of ailment, the nature of the disease being treated, the severity associated with delayed treatment, etc.

From this pool of candidate patients, the method 2000 is configured to predict efficacy and identify one or more candidate traits likely to experience a positive or negative outcome from the proposed treatment. In step 2020, one or more positive outcome (SUCCESS={ }) and negative outcome (FAILURE={ }) data sets are initialized. In this step, a plurality of empty data sets (e.g., SUCCESS and FAILURE data sets) may be created for categorizing one or more patients based on one or more predicted treatment outcomes. Data structures may be prepared for one or more efficacy assessments. Upon having initialized the positive and negative outcomes, at step 2022, a proposed medication or treatment may be temporarily added to one or more patient records. For each candidate patient (xᵢ∈C) remaining after the previous steps, a respective patient medical record may be temporarily augmented with the proposed medication or treatment. The temporarily augmented patient medical records may be analyzed for an efficacy prediction.

Additionally, at step 2022, efficacy of the proposed medication or treatment may be predicted using machine learning models. That is, one or more trained machine learning models (e.g., LLMs with patient graph structures or trained with ailment-specific data (e.g., cancer-related data, chronic care data, antibiotic treatment data, etc.)) may be applied to the augmented patient medical records. It is within the scope of this invention to predict efficacy as previously disclosed in this invention or via any number of the known-in-the-art machine learning based techniques. Both an efficacy and a strength of prediction may be determined for the proposed medication or treatment, using, for instance, one or more methods described herein. In some instances, Rotary Position Encoding (RoPE) may be utilized for temporal and relational aspects of a particular medical event. Severity of ailment or dosage and duration of prescribed medications or treatments may likewise be encoded. RoPE encodes positional information in transformer-based models, so as to represent the order of words or tokens in a sequence. In some embodiments of the method, one or more graphs used to train the LLM are represented using RoPE. In some embodiments, these graphs represent patient medical histories. In some embodiments, these patient medical histories are lifelong histories that contain all medically related events including prescriptions, laboratory readings, doctor visits, prognoses, hospitalizations, surgeries, etc. As related to the methods described herein, RoPE enables models to capture the relative distances between tokens, namely the encoded medical events. Each patient is then categorized into SUCCESS or FAILURE based on predicted efficacy probability.

The method may be further configured to identify positive and negative traits of one or more candidates in the remaining cohort (C). At step 2024, the method 2000 identifies positive traits (Tₚ) (features common to SUCCESS patients) and negative traits (Tₙ) (features common to FAILURE patients) using one or more data analysis techniques such as clustering analysis, association-rule mining, and statistical correlation. Traits may encompass demographics, genetic profiles, biomarkers, comorbidity patterns, and treatment histories.

A SUCCESS set may be analyzed to identify positive traits (Tₚ) common to successful treatment outcomes. A FAILURE set may be analyzed to identify negative traits (Tₙ) common to treatment failures. One or more patient characteristics that correlate with treatment success or failure may then be determined. At step 2026, all remining cohort selection computations and recordings are performed. For example, the determined positive (Tₚ) and negative (Tₙ) traits, the logged original and each successive step removed cohort candidates, and the final selected candidates, are verified and stored. At step 2028 a cohort selection report may be generated that includes some or all of the aforementioned step 2026 stored items. The generated patient cohort report may identify one or more selected trial participants (e.g., the selected cohort) based on the identified positive and negative traits.

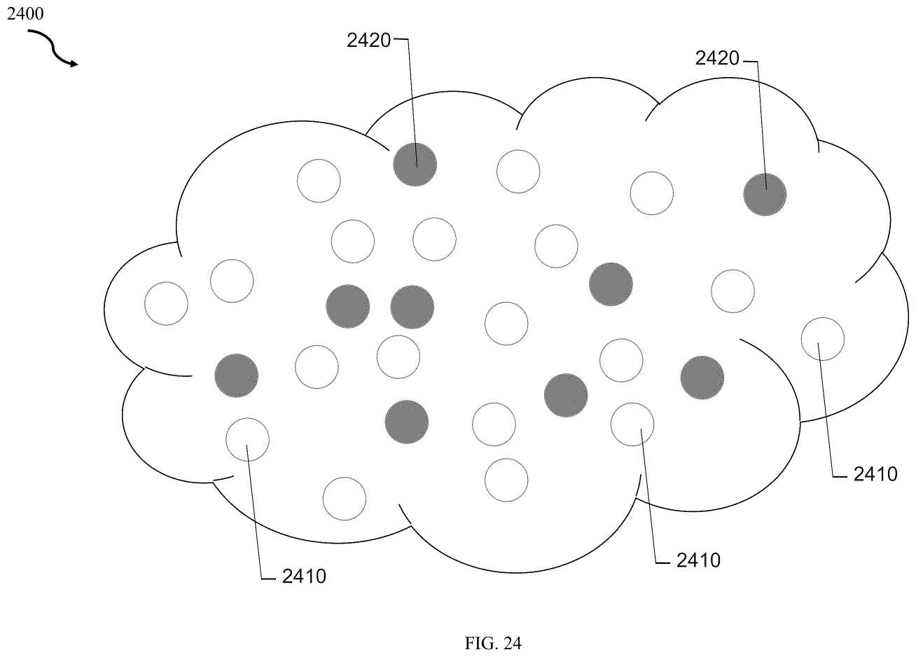

FIG. 24 illustrates an example cohort set (C) 2400 after having identified one or more positive and/or negative traits. The patient cohort report may output one or more cohort selection recommendations (represented by the unfilled circles (e.g., circles 2410) for clinical trial implementation. The final patient cohort includes those who: (i) have the target ailment and satisfy demographic, conflict of interest, historical, and logistic preconditions, (ii) lack a personal or digital-twin history of SAE or EAE in any evaluated category, and (iii) possess one or more positive traits (Tₚ) while lacking negative traits (Tₙ) (represented by filled circles 2420). The report may be further configured to provide recommendations and data visualizations for clinical trial planning and regulatory documentation. In some embodiments, all filtering steps are recorded using non-immutable, durable and permanent logging mechanisms. In some embodiments, blockchain encoding techniques, computed using any of the known-in-the-art computation mediums, might be used. In some embodiments, all computations occur in a cloud-based configuration.

An exemplary block diagram of a computer system 2500, in which processes involved in the embodiments described herein may be implemented, is shown in FIG. 25. Computer system 2500 may be implemented using one or more programmed general-purpose computer systems, such as embedded processors, systems on a chip, personal computers, workstations, server systems, and minicomputers or mainframe computers, or in distributed, networked computing environments. Computer system 2500 may include one or more processors (CPUs) 2502A-2502N, input/output circuitry 2504, network adapter 2506, and memory 2508. CPUs 2502A-2502N execute program instructions in order to carry out the functions of the present communications systems and methods. Typically, CPUs 2502A-2502N are one or more microprocessors, such as an INTEL CORE® processor. FIG. 25 illustrates an embodiment in which computer system 2500 is implemented as a single multi-processor computer system, in which multiple processors 2502A-2502N share system resources, such as memory 2508, input/output circuitry 2504, and network adapter 2506. However, the present communications systems and methods also include embodiments in which computer system 2500 is implemented as a plurality of networked computer systems, which may be single-processor computer systems, multi-processor computer systems, or a mix thereof.

Input/output circuitry 2504 provides the capability to input data to, or output data from, computer system 2500. For example, input/output circuitry may include input devices, such as keyboards, mice, touchpads, trackballs, scanners, analog to digital converters, etc., output devices, such as video adapters, monitors, printers, etc., and input/output devices, such as, modems, etc. Network adapter 2506 interfaces device 2500 with a network 2510. Network 2510 may be any public or proprietary LAN or WAN, including, but not limited to the Internet.

Memory 2508 stores program instructions that are executed by, and data that are used and processed by, CPU 2502 to perform the functions of computer system 2500. Memory 2508 may include, for example, electronic memory devices, such as random-access memory (RAM), read-only memory (ROM), programmable read-only memory (PROM), electrically erasable programmable read-only memory (EEPROM), flash memory, etc., and electro-mechanical memory, such as magnetic disk drives, tape drives, optical disk drives, etc., which may use an integrated drive electronics (IDE) interface, or a variation or enhancement thereof, such as enhanced IDE (EIDE) or ultra-direct memory access (UDMA), or a small computer system interface (SCSI) based interface, or a variation or enhancement thereof, such as fast-SCSI, wide-SCSI, fast and wide-SCSI, etc., or Serial Advanced Technology Attachment (SATA), or a variation or enhancement thereof, or a fiber channel-arbitrated loop (FC-AL) interface.

The contents of memory 2508 may vary depending upon the function that computer system 2500 is programmed to perform. In the example shown in FIG. 25, exemplary memory contents are shown representing routines and data for embodiments of the processes described above. However, one of skill in the art would recognize that these routines, along with the memory contents related to those routines, may not be included on one system or device, but rather may be distributed among a plurality of systems or devices, based on well-known engineering considerations. The present communications systems and methods may include any and all such arrangements.

In the example shown in FIG. 25, memory 2508 may include classifier build and train routines 2512, prediction routines 2514, graph creation routines 2516, kernel feature vector routines 2518, probability routines 2520, and operating system 2522. Classifier build and train routines 2512 may include software to build and train a classifier with an MGKF framework for each type of disease, as described above. Prediction routines 2514 may include software to use the trained classifiers with an MGKF framework to perform prediction for each type of disease, as described above. Graph creation routines 2516 may include software to create patient graphs, as described above. Kernel feature vector routines 2518 may include software to calculate three types of kernel feature vectors, a Temporal Proximity Kernel, a Shortest Path Kernel, and a Node Kernel, between each patient graph and all training examples, as described above. Probability routines 2520 may include software to generate a probability output using the MGKF or an LLM-based approach with same disease diagnosis type, as described above. Operating system 2522 may provide additional system functionality.

As shown in FIG. 25, the present communications systems and methods may include implementation on a system or systems that provide multi-processor, multi-tasking, multi-process, and/or multi-thread computing, as well as implementation on systems that provide only single processor, single thread computing. Multi-processor computing involves performing computing using more than one processor. Multi-tasking computing involves performing computing using more than one operating system task. A task is an operating system concept that refers to the combination of a program being executed and bookkeeping information used by the operating system. Whenever a program is executed, the operating system creates a new task for it. The task is like an envelope for the program in that it identifies the program with a task number and attaches other bookkeeping information to it. Many operating systems, including Linux, UNIX®, OS/2®, and Windows®, are capable of running many tasks at the same time and are called multitasking operating systems. Multi-tasking is the ability of an operating system to execute more than one executable at the same time. Each executable is running in its own address space, meaning that the executables have no way to share any of their memory. This has advantages, because it is impossible for any program to damage the execution of any of the other programs running on the system. However, the programs have no way to exchange any information except through the operating system (or by reading files stored on the file system). Multi-process computing is similar to multi-tasking computing, as the terms task and process are often used interchangeably, although some operating systems make a distinction between the two.

The present invention may be a system, a method, and/or a computer program product at any possible technical detail level of integration. The computer program product may include a computer readable storage medium (or media) having computer readable program instructions thereon for causing a processor to carry out aspects of the present invention. The computer readable storage medium can be a tangible device that can retain and store instructions for use by an instruction execution device.

The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

What is claimed is:

1. A computer-implemented method for selecting a patient cohort for a clinical trial comprising:
retrieving, via a processor and from a secondary storage device storing an electronic medical repository, one or more candidate medical records;
analyzing, via a processor, historical patient data for an initial population of candidate patients eligible for the clinical trial;
identifying, via the processor, one or more adverse effect patient characteristics associated with having a severe adverse effect (SAE) or excessive adverse effect (EAE) for the proposed treatment;
removing, from the initial population of candidate patients and via the processor, any candidate patients identified as having the one or more adverse effect patient characteristics to produce a first subset of eligible candidate patients;
identifying, via the processor, one or more negative outcome patient characteristics associated with one or more negative treatment outcomes for the proposed treatment;
removing, from the first subset of eligible candidate patients and via the processor, any candidate patients identified as having the one or more negative outcome patient characteristics to produce a second subset of eligible candidate patients;
identifying, via the processor, one or more positive outcome patient characteristics associated with one or more positive treatment outcomes for the proposed treatment; and
returning, via the processor, a third subset of eligible candidate patients based on the first and second removing steps and the second identifying step.

2. The method of claim 1, wherein analyzing historical patient data comprises analyzing at least one of a patient medical record, a patient treatment history, or a patient adverse event report for each candidate patient in the initial patient population.

3. The method of claim 1, further comprising:
identifying one or more digital twin patients having one or more similar medical characteristics to at least one candidate patient in the initial patient population; and
removing, via the processor, one or more candidate patients having at least one digital twin reported to have experienced at least one adverse event from the proposed treatment.

4. The method of claim 3, wherein the at least one adverse event experienced by the at least one digital twin was a severe adverse effect (SAE).

5. The method of claim 3, wherein the one or more digital twin patients are identified based on a similarity between the one or more digital twin patients and at least one candidate patient in at least one of: demographic characteristics, medical history, genetic markers, biomarkers, or comorbidities.

6. The method of claim 5, wherein the identification of the one or more digital twins is determined, via a processor, using graph matching properties.

7. The method of claim 6, wherein the graph matching properties used rely on patient demographic characteristics stored within one or more nodes in a patient graph.

8. The method of claim 3, wherein analyzing the one or more digital twin patients comprises examining one or more adverse events experienced by at least one of the digital twin patients when exposed to: (i) the proposed treatment as a treatment for one or more other conditions, (ii) one or more other treatments for the same or similar conditions, or (iii) one or more treatments administered via a same delivery method as the proposed treatment.

9. The method of claim 1, wherein identifying one or more negative outcome patient characteristics comprises determining one or more patient traits that correlate with treatment failure or poor treatment efficacy.

10. The method of claim 1, wherein identifying one or more positive outcome patient characteristics comprises determining one or more patient traits that correlate with treatment success or high treatment efficacy.

11. The method of claim 1, wherein the third subset of candidate patients comprises one or more candidate patients having the one or more positive outcome patient characteristics and excludes one or more patient candidates having at least one of the one or more adverse effect patient characteristics or the one or more negative outcome patient characteristics.

12. The method of claim 1, further comprising predicting treatment outcomes for one or more candidate patients using a machine learning model trained on the historical patient data.

13. The method of claim 12, wherein the machine learning model comprises a large language model (LLM) trained on one or more patient graph structures corresponding to at least one of the candidate patients in the initial patient population representing at least one of a medical event or a treatment outcome for the at least one candidate patient.

14. The method of claim 13, wherein the LLM uses Rotary Position Encoding (RoPE) to represent one or more of temporal and relational aspects of the medical event or the treatment outcome.

15. The method of claim 13, wherein the LLM is further trained using at least one of: ailment-specific data corresponding to the target condition or medication delivery-specific data corresponding to the delivery method of the proposed treatment.

16. The method of claim 1, wherein identifying patient characteristics comprises applying data analysis techniques selected from at least one of clustering analysis, association rule mining, or statistical analysis.

17. The method of claim 1, further comprising filtering the initial patient population based on predetermined demographic criteria and presence of a target ailment.

18. A system for clinical trial patient cohort selection comprising:
a processor; and
a memory storing instructions that, when executed by the processor, cause the system to:
retrieve one or more candidate medical records from an electronic medical repository;
identify one or more adverse effect patient traits associated with an adverse effect risk for a proposed treatment;
determine one or more positive patient traits associated with a positive outcome for the proposed treatment;
determine one or more negative patient traits associated with a negative outcome for the proposed treatment;
generate a patient cohort report based on the one or more positive patient traits; and
output the generated patient cohort report for clinical trial implementation.

19. The system of claim 18, wherein the memory further stores instructions that cause the system to identify digital twin patients and analyze adverse events experienced by the digital twin patients to inform the identification of adverse effect patient traits.

20. A non-transitory computer-readable medium storing instructions that, when executed by a processor, cause the processor to perform a method for clinical trial cohort selection comprising:
retrieving one or more candidate medical records from an electronic medical repository;
identifying one or more adverse effect patterns and one or more treatment efficacy patterns for a proposed therapy;
determining one or more patient characteristics predictive of treatment success or treatment failure;
excluding one or more trial participants based on the determined treatment failure characteristics;
selecting one or more trial participants based on success-predictive patient characteristics; and
generating a cohort selection report identifying the selected one or more trial participants.
